(19) United States
(12) Patent Application Publication
(10) Pub. No.: US 2025/0002903 A1
(43) Pub. Date: Jan. 2, 2025

(54) Engineering Bacteria Swarm Patterns for Spatiotemporal Information Encoding

(71) Applicant: The Trustees of Columbia University in the City of New York, New York, NY (US)

(72) Inventors: Tal DANINO, Brooklyn, NY (US); Anjali DOSHI, New York, NY (US); Marian SHAW, New York, NY (US); Andrew LAINE, New York, NY (US); Jia GUO, Fort Lee, NJ (US)

(73) Assignee: The Trustees of Columbia University in the City of New York, New York, NY (US)

(21) Appl. No.: 18/746,091

(22) Filed: Jun. 18, 2024

Related U.S. Application Data
(63) Continuation of application No. PCT/US2022/053409, filed on Dec. 19, 2022.
(60) Provisional application No. 63/301,182, filed on Jan. 20, 2022.
(60) Provisional application No. 63/291,423, filed on Dec. 19, 2021.

(51) Int. Cl. C12N 15/10

(52) U.S. Cl. CPC C12N 15/1086; C12N 15/1089

(57) ABSTRACT

Encoding environmental parameters by modulating swarm patterns of bacteria on substrates and correlating spatiotemporal changes in the environmental parameters to changes in swarm patterns using one or more trained machine learning models.

### CROSS-REFERENCE TO RELATED APPLICATIONS

This is a continuation of International Patent Application No. PCT/US2022/053409 filed on 19 Dec. 2022, which claims priority to U.S. Provisional Patent Applications Nos. 63/291,423 filed on 19 Dec. 2021 and 63/301,182 filed 20 Jan. 2022, each of which are incorporated herein in their entirety.

### STATEMENT REGARDING FEDERALLY SPONSORED RESEARCH OR DEVELOPMENT

This invention was made with government support under grants 1644869 and 1847356 awarded by the National Science Foundation. The government has certain rights in the invention.

### TECHNICAL FIELD OF THE INVENTION

This disclosure generally relates to the field synthetic biology. More specifically, the disclosure relates to encoding environmental parameters using bacteria that form visible swarming patterns on substrates (e.g., engineered Proteus mirabilis) and detecting spatiotemporal changes in those environmental parameters by detecting differences between observed and expected swarm patterns using macroscopic images of the swarm patterns and one or more trained machine learning models.

### BACKGROUND OF THE INVENTION

Many bacterial species form complex spatial patterns on solid agar surfaces via swarming motility, a flagella-powered rapid coordinated movement of bacteria across a surface. The unique swarming of Proteus mirabilis, a commensal gut bacterium also commonly found in soil and water, produces a “bullseye” pattern on solid agar defined by concentric rings of high bacteria density that are visible to the naked eye. This pattern is created from a sequence of phases starting with initial colony growth (lag), followed by oscillatory cycles of synchronized colony expansion (swarming), and stationary periods of cell division (consolidation). The synchrony of the bacterium's swarming is achieved by complex coordination of cell elongation, secretion of surfactant to aid movement, intercellular communication, and alignment of swarmer cells into rafts by intercellular bundling of overexpressed flagella.

Within the synthetic biology field, there have been recent efforts towards microbial information recording, primarily through use of DNA for long term storage, multiplexed recording of external inputs, and temporally resolved signal recording cells, but this form of information encoding requires sequencing for information recovery. Accordingly, an alternative way of recording information that would avoid this sequencing limitation would be highly desirable. While the ability of P. mirabilis to produce patterns has been known for over one hundred years, this behavior has yet to be engineered for biotechnological applications, such as recording information about environmental conditions.

### BRIEF SUMMARY OF THE INVENTION

The present disclosure relates to methods of detecting spatiotemporal changes in an environmental parameter (e.g., temperature, humidity, pH, etc.) by recording visual patterns produced by bacteria that naturally exhibit swarming patterns (e.g., P. mirabilis, P. aeruginosa, Paenibacillus vortex, B. subtilis str. 3610, and P. aeruginosa PA14) over time, and detecting differences between observed and expected swarm patterns at various time points using one or more trained machine learning models.

In some embodiments, bacteria are transgenic in nature and comprise at least one exogenous inducible promoter that controls at least one gene related to swarming motility exhibited by the bacteria. In some embodiments, the inducible promotor is regulated directly or indirectly by the environmental parameter. In some embodiments, the inducible promoter is induced by an agent, such as isopropyl ß-D-1-thiogalactopyranoside (IPTG) and arabinose. In some embodiments, the at least one gene is selected from the group consisting of cheW, fliA, flgM, umoD, and lrp.

Spatiotemporal changes in the environmental parameter are detected by analyzing differences between an observed swarming pattern and an expected swarming pattern of the bacteria using one or more trained machine learning models. In some embodiments, these methods of analysis comprise receiving at least one image of a plurality of bacteria configured in a macroscopic orientation and recognizing, within the image, a visual pattern of the plurality of bacteria and generating a mask that approximates the visual pattern of the plurality of bacteria using one or more trained machine learning models.

In some embodiments, the visual pattern comprises at least one ring. In some embodiments, the visual pattern comprises an outer ring and a plurality of inner rings. In some embodiments, the visual pattern comprises a bullseye pattern.

In some embodiments, the mask is generated by combining outputs from a first machine learning model and a second machine learning model. In some embodiments, the first machine learning model is configured to generate a mask that approximates a first portion of the visual pattern and the second machine learning model is configured to generate a mask that approximates a second portion of the visual pattern. In some embodiments, the first machine learning model and the second machine learning model comprise convolutional neural networks. In some embodiments, the first machine learning model is configured to detect an outer ring of the visual pattern and generate a mask that approximates the outer ring, and the second machine learning model is configured to detect inner rings of the visual pattern and generate a mask that approximates the inner rings. In some embodiments, the first machine learning model is configured to detect the outer ring of the visual pattern by segmenting the image into multiple patches and distinguishing the outer ring from background in each patch.

In some embodiments, the present disclosure also relates to a system for performing macroscopic analysis of a plurality of bacteria, comprising (a) a processor; and (b) a memory storing instructions for execution by the processor, the instructions configuring the processor to (i) receive at least one image of the plurality of bacteria configured in a macroscopic orientation; and (ii) recognize, within the image, a visual pattern of the plurality of bacteria and generate a mask that approximates the visual pattern of the bacterial colonies using one or more trained machine learning models.

In some embodiments, the present disclosure also relates to a non-transitory computer readable medium having instructions stored thereon that, when executed by a processor, cause the processor to perform macroscopic analysis of a plurality of bacteria, wherein, when executed, the instructions cause the processor to: (i) receive at least one image of a plurality of bacteria configured in a macroscopic orientation; and (ii) recognize, within the image, a visual pattern of the plurality of bacteria and generate a mask that approximates the visual pattern of the plurality of bacteria using one or more trained machine learning models.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description of the Invention. This summary is not intended to identify key features or essential features of the claimed subject matter, nor is it intended to be used as an aid in determining the scope of the claimed subject matter.

### BRIEF DESCRIPTION OF THE SEVERAL VIEWS OF THE DRAWINGS

FIGS. 1A-1F show how P. mirabilis swarming is manipulated for spatio-temporal information encoding. (A) Wild type P. mirabilis cells undergo oscillatory swarming on solid agar to grow into a characteristic bullseye colony. P. mirabilis is engineered with an externally inducible circuit driving swarming-related genes to modify the macroscale pattern output, which can then be decoded using quantitative methods to predict the input conditions. (B) Colony patterns formed by a strain with a control circuit with GFP (top) compared with a circuit with the chemotaxis gene cheW (bottom), grown on agar supplemented with various IPTG concentrations. (C) The colony pattern is distilled into radially averaged pixel intensity trajectories, with distinct peaks matching low-density ring boundaries when plotted as a heatmap or line plot. The blue line denotes the mean trajectory of the individual plates (gray). (D) Heatmaps of average cheW trajectories at varying IPTG concentration (n=5 plates at each condition except 0 IPTG (n=6)). (E) Radii of the colonies plotted by IPTG concentration after 24 hours (filled circles) and approximate ring width (empty triangles), calculated from Fourier analysis of the radially averaged trajectories. The mean and standard error of the mean (SEM) are shown in black. (F) A multinomial model was fit to the measurements in e, with predicted IPTG concentration as the output variable. The model's predictions for each plate shown in e are shown as a confusion matrix.

FIGS. 2A-2M illustrate the modulation of swarm colony parameters in engineered P. mirabilis. (A) Candidate genes involved in swarming pathway were chosen for construction of inducible strains. The patterns in the presence of inducer were characterized by swarm assays, then by specific feature measurements used to recover the inducer concentration. (B) Characteristic patterns of engineered strains in the presence of IPTG and closeups of pattern features. (C) Range of patterns formed. PM7002 with indicated inducible swarm plasmids was grown for 24 hours at a range of IPTG concentration. Representative images of three replicates are shown. (D) Closeups of patterns formed at 0 mM IPTG. (E) Petri dishes were scanned at high resolution. The Petri rim was identified and cropped out using MATLAB functions (see Methods). Images were thresholded to show only the colony inoculum and the center point was identified using MATLAB functions. Images were converted from Cartesian to polar coordinates with interpolation, and the flattened images were used for subsequent analysis. (F) For each induced strain, heatmaps and plots of radially averaged intensity trajectories across the colony radius for the representative images in (B). (G) Fourier transforms of the polar images visualize the intensity frequencies of each induced strain. (H) Proportion of information contained in central region of Fourier transform of the polar pattern image to total magnitude of the Fourier image is plotted at a range of IPTG conditions. (I) Quantification of aspects of colony patterns of engineered strains at increasing IPTG concentrations, measured as in FIGS. 2H, K and L. All strains had at least n=3 plates measured at each IPTG concentration. Error bars represent standard error of the mean. (J) Multinomial regression model performance on engineered strains. Each flattened image was divided into four sectors (each 250 columns wide) to augment number of measurements available and allow model to converge. Measurements listed in c were obtained on each sector and mnrfit function was used to obtain a best fit model for classifying sets of measurements into bins 1: 0-0.9 mM IPTG, 2: 1-5 mM IPTG, 3: 5-10 mM IPTG. Confusion matrices were then plotted; numbers on the matrices represent numbers of plates. Calculated AUC values ranged from 0.60 to 0.965. (K) Local radial coefficient of variation (CV), which increases with colony asymmetry. (L) Change in intensity from the densest edge of the inoculum to the low-density region immediately surrounding it, i.e., distinctness of the inoculum edge. (M) Area under the curve (AUC) of multinomial regression models fit with measurements and IPTG concentrations for each strain.

FIGS. 3A-3H show the dynamics of engineered P. mirabilis strains. (A) Time-lapse of P. mirabilis with inducible umoD expression or inducible GFP expression (control). Plates contained 20 mL 1.3% agar with 10 mM IPTG. (B) Heatmap visualizations of swarming pattern development from center of plates (0 cm on left axis) to edge (top and bottom edges) for each image in the time-lapses in (A). Radially averaged pixel intensity, a proxy for local colony density, at each location on plate is represented by heatmap color, with blue indicating least dense and yellow indicating most dense regions. Active regions and time periods of colony expansion via swarming are visible as faint blue diagonal edges. Consolidation phases appear as horizontal edges corresponding with increasing density (darker colors) within the colony. (C) Colony front distance from center plotted as a function of time for a single time-lapse of six plates. All plates contained 10 mM IPTG. (D) Mean lag, swarm, and consolidation times calculated from the trajectories in (D). (E) Mean of the swarm speeds for each strain in the same time-lapse. (F) Tuning dynamics of P. mirabilis pattern formation by strain. Timelapse growth of various engineered P. mirabilis strains at varying IPTG concentrations are displayed as heatmaps, with blue representing lightest pixel intensity (least dense) and yellow representing densest colony portions. The colony front can be identified as a faint light-colored line emanating outwards from the initial center inoculum (horizontal bar across each heatmap). (G) Plots of dynamic characteristics of engineered strains vs IPTG concentration, measured from one to two timelapses per condition per strain. Individual dots represent individual timelapses (lag times) or individual phases in all timelapses (consolidation and swarm phase lengths, swarm speeds). Lines represent the mean of these individual measurements. Middle swarm or consolidation phase lengths were determined by discarding the measurements of the first and last of these phases for each timelapse, or discarding the last phase if the timelapse had only two of the given phase. (H) The local CV of the swarm front, averaged over the plate and averaged over at least 2 timelapses per IPTG concentration. Error bars represent SEM.

FIGS. 4A-4O illustrate multi-condition pattern encoding and deep-learning models for decoding. (A) pBADumoD control. Representative images of colonies after 24 hours of growth on 1.5% agar with indicated arabinose concentrations are shown. Plots of the mean area (percent of plate) and average radial coefficient of variation of cheWumoD patterns; error bars represent standard error of the mean. For colony area, comparing 0.1% to 0.2% arabinose, p=0.0145 for two-sample t-test at 2.5 mM IPTG, but at 0 IPTG p=0.2235 and at 5 IPTG p=0.7. (B) Dual input swarming strain with IPTG-inducible expression of cheW and arabinose-inducible expression of umoD. (C) Representative images of colony patterns produced by the dual cheW/umoD strain in response to combinations of IPTG and arabinose. (D) Heatmaps of radially averaged profiles of the patterns in (C) are shown. (E)-(F). Mean colony area (calculated as percent of agar pixels in flattened image) and coefficient of variation for all plates (n>=14) at each combination of IPTG and arabinose. (G) Training and validation accuracy and loss for a CNN model which had three convolutional/max pooling blocks, trained on the dataset of images of the combination strain at various IPTG and arabinose conditions. (H) Fine tuning of three architectures pre-trained on ImageNet weights; right hand panels represent models' ability to identify the correct image class within its top three predicted classes. (I) Representative images of pLacflgM strain pattern at the given conditions. The difference between incubator and benchtop patterns is clearer at 10 mM IPTG. (J) Training and validation accuracy and recall of the U-Net model with VGG-11 encoder, training on 18 images. (K) Confusion matrix for the InceptionV3 model's accuracy of predicting combinations of IPTG and arabinose concentrations from endpoint patterns unseen during training. Total available images per class shown below matrix; an 80/20 train/test split was used. Numbers on matrix represent fraction of test images per true class (L). Visualization of the pixel attributions from the InceptionV3 model for representative, correctly-predicted images of each class. Darker orange represents higher weight of that pixel on the final prediction. (M). Schematic of encoding of changes of environment within developing flgM pattern. (N) Example patterns of the flgM strain grown with 10 mM IPTG and moved between the benchtop and incubator. Arrows mark boundaries between regions of the pattern formed in different conditions, i.e., the location of the colony edge at the time of a switch in conditions. (O) Examples of the predicted boundary masks generated by the trained U-Net compared with ground truth annotations for the pattern images shown in (N), which were unseen during training.

FIGS. 5A-5I show the engineering of metal-sensing strains of P. mirabilis. (A)-(C) show the maximum fold change of GFP, expressed from either pCopA or pCadA, achieved over the course of 17 hours at the indicated concentration normalized to uninduced wells, in plate reader. (D) Schematic of copper-sensing strain pCopA-flgM. (E) Location of spots in plate images. Representative images of colonies of indicated strains with added spots of copper at the indicated concentrations. (F) Mean middle ring widths on the sides of the plates with added copper, normalized to the mean width for the same-day 0 copper plates of the same strain. Individual points represent separate plates. Error bars represent standard deviation. From top to bottom: P=2.5e-5, 1.3e-6, 0.1 (n.s.), 3.9e-5, 6.3e-9, 2.3e-13. P values were calculated by one-way ANOVA. (G) Colony radius of the copper-induced side of the plates, as shown in (D). (H) shows the mean of the middle ring widths (i.e., neither innermost nor outermost) of the colonies on the sides with copper. Each measurement represents a separate plate. (I) shows the same measurements normalized to the same-day GFP measured mean widths at the given concentration.

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes only and are not to be construed as limiting the subject matter described.

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

In addition, ranges provided in the specification and appended claims include both end points and all points between the end points. Therefore, a range of 1.0 to 2.0 includes 1.0, 2.0, and all points between 1.0 and 2.0.

The term “about” as used herein when referring to a measurable value such as an amount, a temporal duration, and the like, is meant to encompass variations of .+−.20%, .+−.10%, .+−.5%, .+−.1%, or .+−.0.1% from the specified value, as such variations are appropriate to perform the disclosed methods.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or lists of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of “consisting essentially of,” when used in the claims, shall have its ordinary meaning as used in the field of patent law.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

The inventions described herein relate to macroscopic analysis of bacterial colonies (e.g., engineered Proteus mirabilis bacteria) using deep learning models to generate masks that approximate visible swarm patterns formed by the bacteria and using the engineered bacteria to encode environmental conditions by modulating the swarm patterns and correlating changes in environmental conditions to changes in swarm patterns using one or more trained machine learning models as described hereinbelow.

### EXAMPLES

The following examples have been included to illustrate aspects of the inventions disclosed herein. In light of the present disclosure and the general level of skill in the art, those of skill appreciate that the following examples are intended to be exemplary only and that numerous changes, modifications, and alterations may be employed without departing from the scope of the disclosure.

### Example 1

### Bacterial Strains and Growth Conditions

P. mirabilis and E. coli were cultured in LB media (Sigma-Aldrich) supplemented with 5 or 50 μg ml⁻¹ kanamycin, respectively. P. mirabilis was grown on either 3% or 1.5% agar to suppress or allow for swarming, except for time-lapse assays as indicated.

### Example 2

### Competent Cell Preparation

P. mirabilis (PM7002) cells and E. coli (Mach1) were made electrocompetent as follows. A fresh 2-mL overnight culture was subcultured 1:100 in 50 mL Luria-Bertani (LB) broth, then grown at 30° C. with shaking until logarithmic growth phase was reached, indicated when the optical density at 600 nm (OD₆₀₀) was 0.4-0.6. Growth was stopped by incubation of the culture on ice for 15 minutes. Cells were then pelleted by centrifuging for 10 minutes at 4° C. and 3000 rpm. After decanting, the pellet was washed three times in either 50 mL ice-cold filter-sterilized 10% glycerol (P. mirabilis) or 50 mL ice-cold filter-sterilized water (E. coli), then resuspended in 220 μL 10% glycerol. 50 μL aliquots were stored in −80° C.

### Example 3

### Strain Construction

The previously constructed plasmid pZE24 (pLacGFP pConstLacIQ) plasmid, containing the ColE1 origin of replication and a kanamycin resistance cassette, was used as the backbone for the inducible swarming plasmids. Plasmids and chromosomal P. mirabilis DNA were prepared using standard procedures (Qiagen). Swarming gene sequences were obtained from GenBank (JOVJ00000000.1) and Gibson primers were designed (Eton) to amplify the genes from the chromosomal DNA via PCR (Phusion). A set of swarming plasmids were constructed using Gibson Assembly and standard restriction digest and ligation cloning to replace the GFP gene with the appropriate swarming gene. For plasmids which additionally contained the pBAD-araC operon, this was obtained from the pBADmCherry-pConstAra plasmid (ATCC54630). After cloning plasmids into Mach1 E. coli, clones were verified via colony PCR (Phusion) and sequencing (Eton). Clones were then grown at 37° C. with shaking overnight before being stored in 50% glycerol at −80° C.

### Example 4

P. mirabilis Transformation

Plasmid DNA was introduced into P. mirabilis competent cells as follows. 50 μL aliquots of competent cells were thawed on ice for 10 minutes. DNA was added to the cells (200-400 ng DNA in a volume of 1-5 μL per aliquot). The mixture was then incubated on ice for one hour. Cells were electroporated in prechilled electroporation 0.1 cm electrode gap cuvettes using a Bio-Rad MicroPulser set to E1 setting (1.8 kV) for bacterial electroporation. Cells were recovered by adding 1 mL prewarmed SOC medium and incubated with shaking at 37° C. for 3 hours. The cells were pelleted by centrifugation for 10 minutes at 4° C. and 3000 rpm, and 700 μl of the supernatant was decanted before resuspension in the remaining 300 μl. The cells were then plated on pre-warmed 3% LB agar plates with antibiotics as necessary and incubated at 37° C. for 22-24 hours. Single colonies were inoculated and fresh overnight cultures were stored in 50% glycerol at −80° C.

### Example 5

### Bacterial Growth and Swarm Assay

Overnight liquid bacterial cultures were prepared by inoculating LB broth with cells from the −80° C. glycerol stocks and supplementing with 50 mg/mL kanamycin as appropriate. Cultures were incubated at 37° C. with shaking for 12-16 hours. The OD₆₀₀ of each culture was measured and normalized to 1.0 by dilution with LB broth. 1.5% agar (or, where indicated, 1.3% agar) was autoclaved, then cooled to 50-55° C. with stirring. 5 mg/mL kanamycin, IPTG and/or arabinose were then added. 15 mL agar was poured in each 100×15 mm Petri dish and left to solidify partially uncovered under an open flame for exactly 30 minutes. 2 μL of the previously diluted liquid culture was inoculated on the center of each Petri dish and dried for 15 minutes partially uncovered under open flame. The plates were incubated at 37° C. for 22-24 hours, then individually imaged using a scanner (Epson Perfection V800 Photo Scanner) set to 48-bit Color and 400 dpi. The scanner was kept on the benchtop and room lighting was similar during all experiments; other settings of the scanner were also kept the same between experiments. Incubator humidity typically varied between 50-80% during the course of experiments.

### Example 6

### Time-Lapses

For time-lapses on the benchtop (room temperature), up to six 1.3% LB agar plates with 20 mL agar were inoculated and placed on the flatbed scanner using the previously described settings, and kept upside down to prevent condensation. A custom AppleScript was written to scan plates every 10 minutes for the course of the time-lapse (48-72 hours).

### Example 7

### Computational Methods

Measurements of colony features were taken using MATLAB (Mathworks) image and signal processing functions. Images were preprocessed by conversion to grayscale, then using the imfindcircles function (based on a Hough transform) and regionprops to identify and remove out the plate rim, then thresholded to find the colony's center inoculum, typically easily identified by its dark boundary. Upon finding the center point, the colony was unrolled or “flattened” using a Cartesian to polar transformation and the scattered interpolant function, and resized to 1000×1000 pixels for comparability between images. The colony rim was also masked out (set to white). Simple analysis of the mean intensity over the plate could be then carried out by averaging the pixel intensity across each row of the image.

Where described, local CV was calculated by moving a sliding window region of width 10 pixels across each row and calculating the CV within it, then taking the average of these calculated CVs over the whole image. Mean CV was calculated by obtaining the CV across each row. The inoculum edge intensity was measured for a given image as follows: the image was smoothed using the movmean function with averaging applied in 25-pixel windows horizontally. For each individual column of the smoothed image, the minimum value between the 15^{th} and 60^{th} rows (i.e., in the region of the inoculum border) was subtracted from the maximum value in that region. The average over all the columns was then taken.

For certain measurements, a mask of the colony region was desired. A custom algorithm was developed using image processing functions in MATLAB. Briefly, a set of filters were applied to reduce local noise such as dust and scratches, then adaptive histogram equalization was applied to increase contrast. The entropyfilt function in matlab was applied and the output was thresholded, then the difference between this output and the original image was taken in order to sharpen the edges in the colony. The image was binarized and a series of morphological operations including dilation, opening, and hole-filling were applied to obtain a mask of the colony. The largest region was retained and all smaller regions were discarded.

In order to analyze the timelapses, a method to track a growing swarm colony was sought. P. mirabilis presents a unique challenge in this area; during its swarm phase, only a thin, almost transparent film of bacteria move outwards, almost indistinguishable from local variations in agar intensity, so the swarm front is difficult to detect with conventional edge-detection algorithms. The described colony region isolation algorithm also did not work on these images. The movement outwards on the plate (or vertically down on the flattened images) over time is difficult and noisy to capture. Towards an algorithm for tracking the swarm edge, each timelapse image was first flattened as described above. Each image was subtracted from the image before it with the imabsdiff function. The difference images were then averaged across columns, creating a radially averaged trajectory. In brief, the findpeaks function was used on each timepoint's trajectory, using a custom algorithm and manual parameter refinement to determine the location in which to seek the peak, and taking advantage of the constraint that the colony edge would not move backwards over time. The obtained colony front trajectory was then labeled using a custom algorithm involving the moving_polyfit function, bwareaopen and bwlabel, from which the locations of the lag phase, swarm phases, and consolidation phases were obtained.

Statistical tests were calculated and data was plotted either in MATLAB or in Python. Latex tables were generated using Overleaf Multinomial regression models were fit to the measurements using the mnrfit function in MATLAB. For the single gene data, each flattened image was divided into four sectors (each 250 pixels wide) and measurements were taken on each sector to increase the number of measurements available, so that the model fitting could converge. The models were evaluated using the multiClassAUC function which implements the Hand and Till function for area under the curve for multi-class problems. Machine learning models were implemented in Tensorflow and Pytorch, with manual annotation of the flgM ground truth segmentation done using the LabelMe program. Attributions were calculated following the Integrated Gradients method of Sundarajan et al.

### Example 8

### Swarm Pattern Modulation

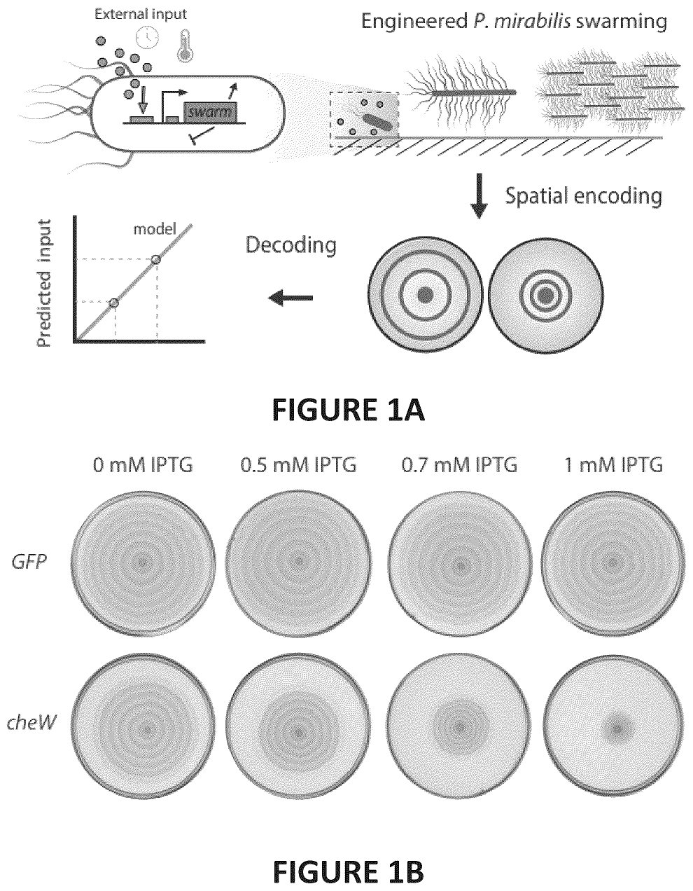

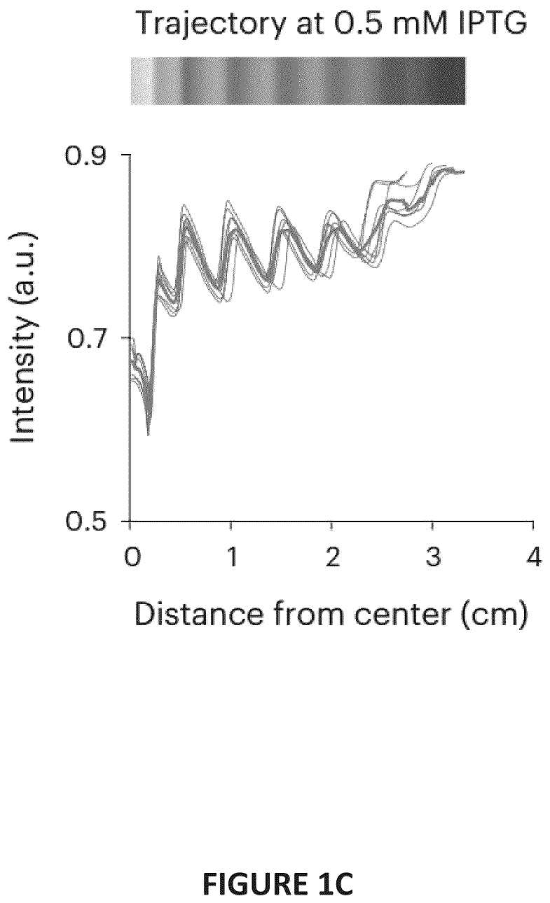

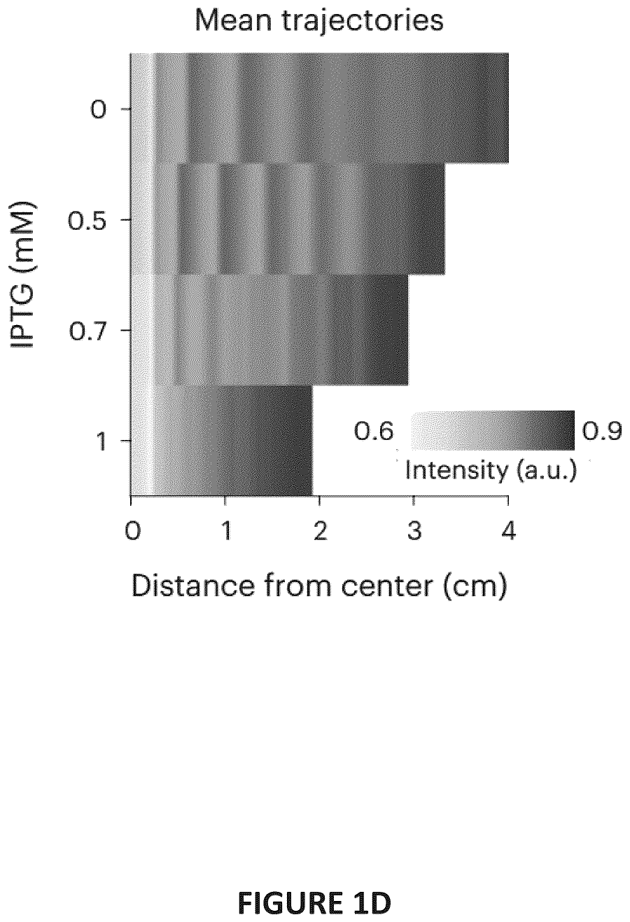

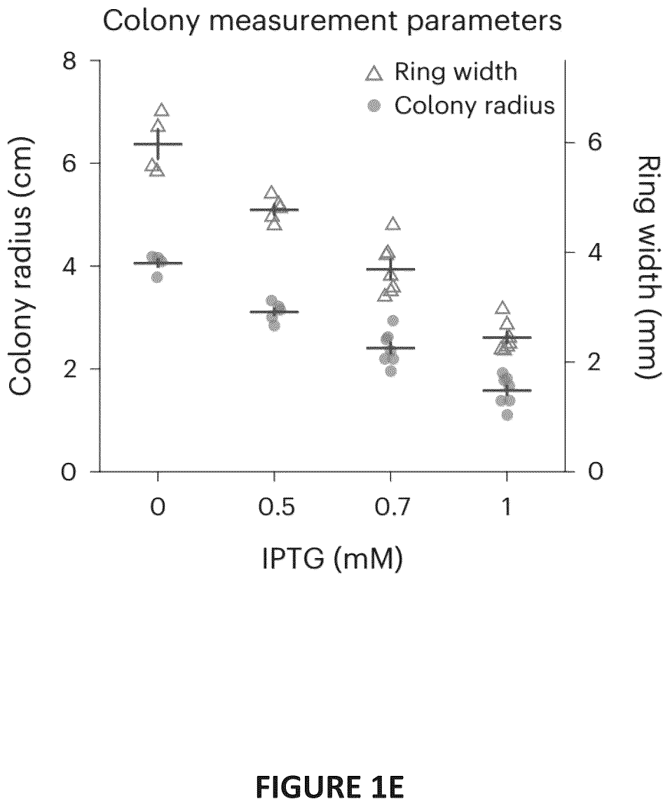

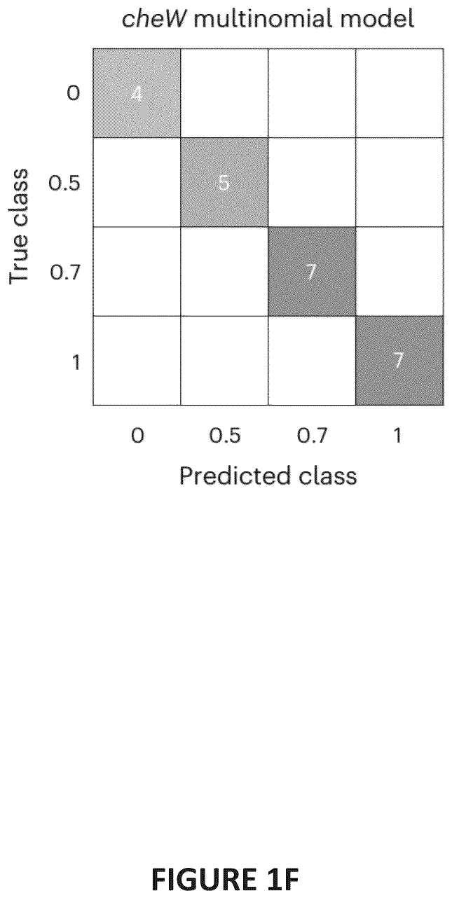

To initially demonstrate swarm pattern modulation, a strain of P. mirabilis (PM7002) were transformed with a promoter induced by isopropyl ß-D-1-thiogalactopyranoside (IPTG) expressing cheW, a chemotaxis-related gene upregulated in the swarming process. Induction of cheW overexpression with increasing concentrations of IPTG generated colonies of decreasing size and ring width as compared to a gfp-expressing control (FIG. 1). To quantify this observation, the radially averaged pixel intensity as a proxy for colony density was examined in each of the conditions (FIG. 1C). All of the colonies had a characteristically dense boundary at the initial inoculum, seen as a dip in the intensity plot, and showed periodic changes in density across the colony. With higher cheW overexpression induced by IPTG, decreased colony radius was accompanied by increased density near the inoculum and denser ring boundaries, seen as lighter areas on heatmaps of radially averaged intensities (FIG. 1D). A small dataset was constructed and the colony radius was manually measured using image processing tools (FIG. 1E). As expected, the radially averaged profiles showed peaks of intensity in the periodic ring boundaries. To measure the ring width, profiles were transformed to the Fourier domain and a low-pass filter was applied to calculate mean ring width based on high-magnitude frequencies (FIG. 1E). The associations between colony radius and ring width correlated well with IPTG concentrations (R²=0.90, 0.90), and using this as a calibration curve for predicting IPTG concentrations resulted in accuracies of ˜92%. To improve upon this concept of decoding input conditions from the pattern, a multinomial regression model was constructed using these measurements; the model correctly predicted each colony's input IPTG from its radius and ring width in all cases (FIG. 1F). It was thus reasoned that colony features could in principle encode information about external inputs the bacteria received, and feature measurements could be used to decode the information.

### Example 9

### Manipulation of Multiple Swarming-Pathway Genes

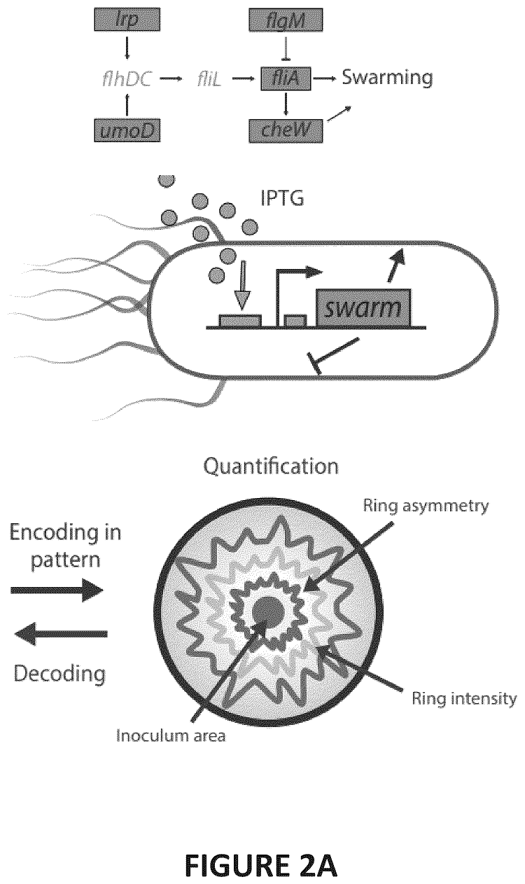

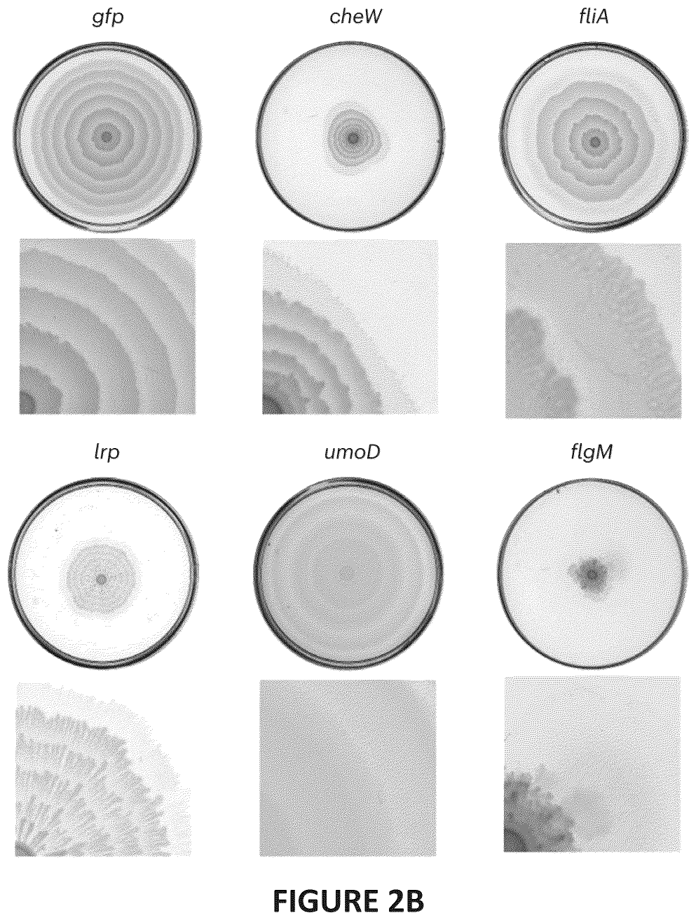

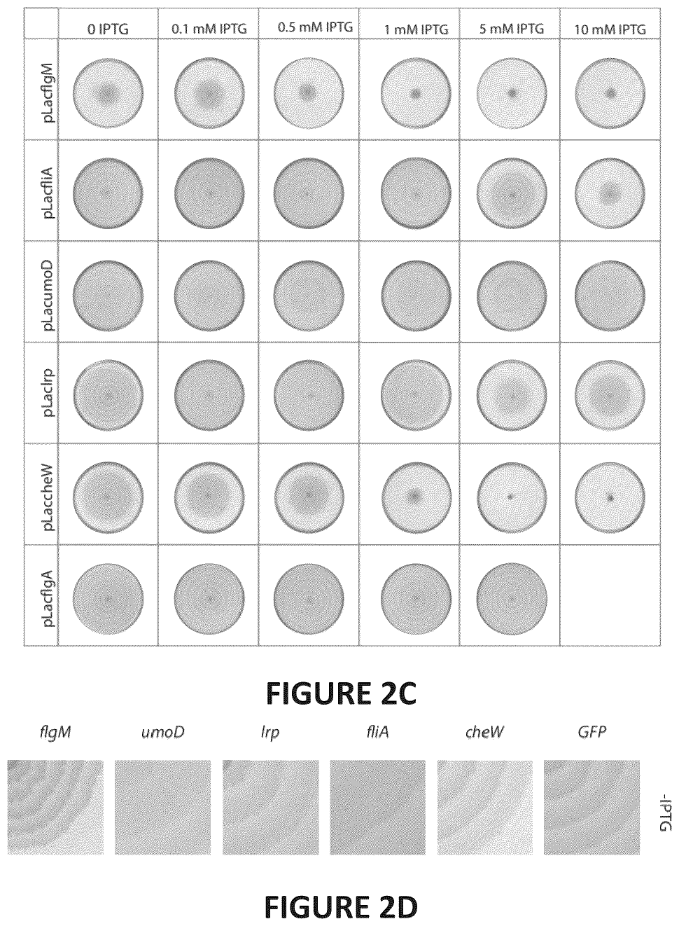

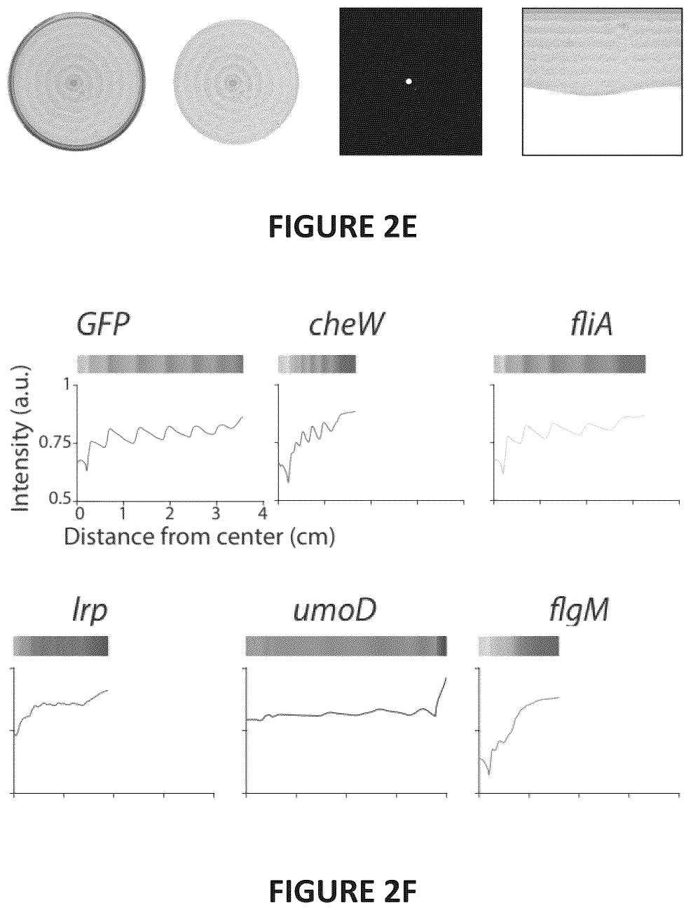

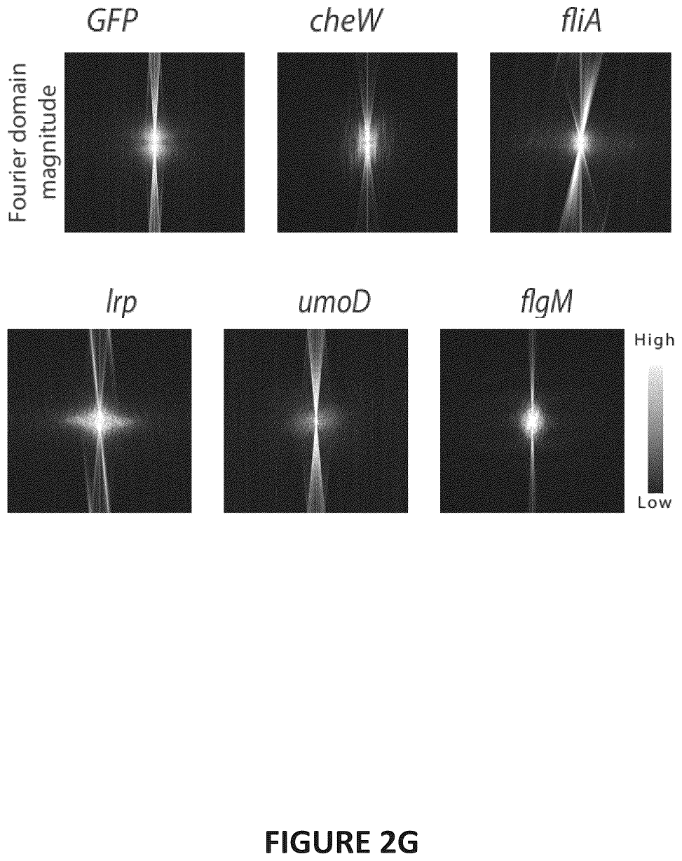

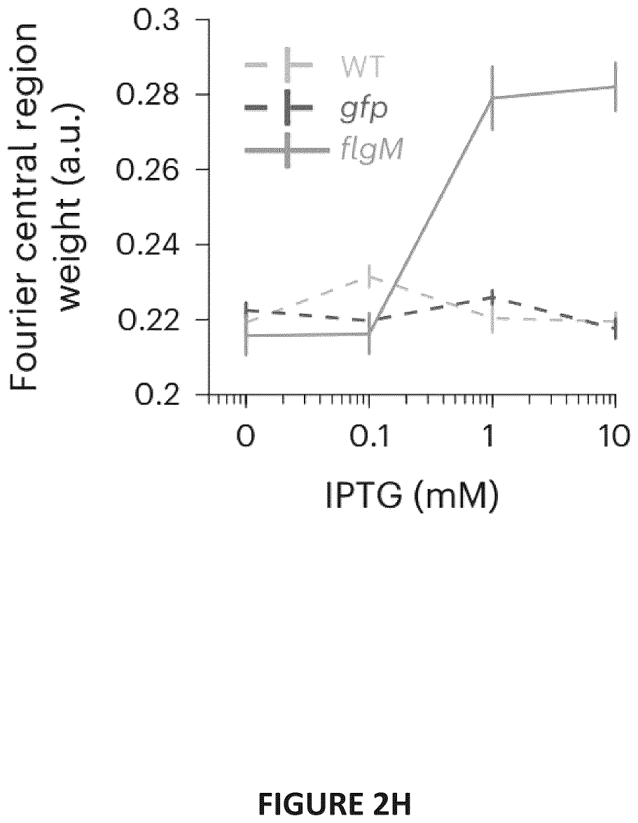

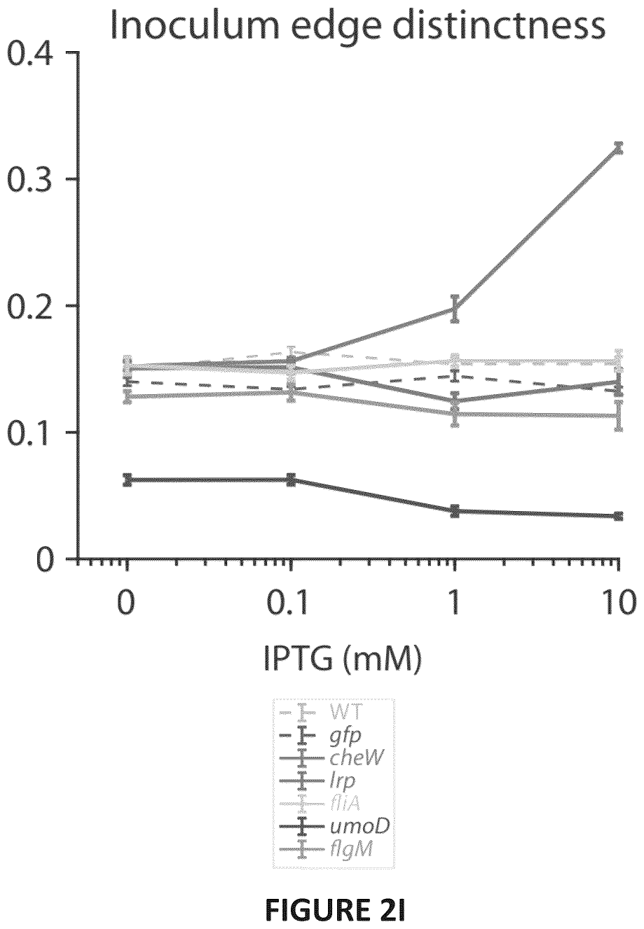

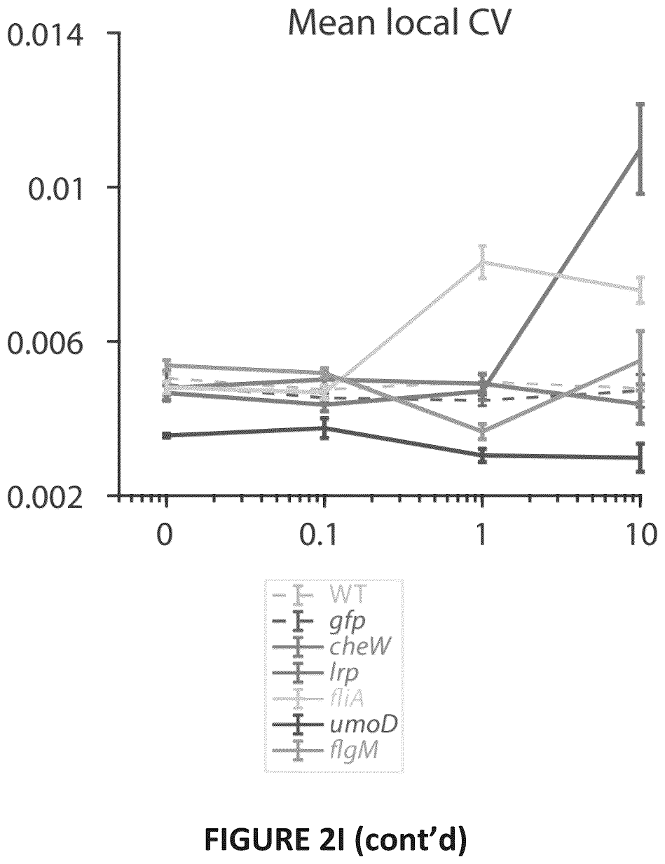

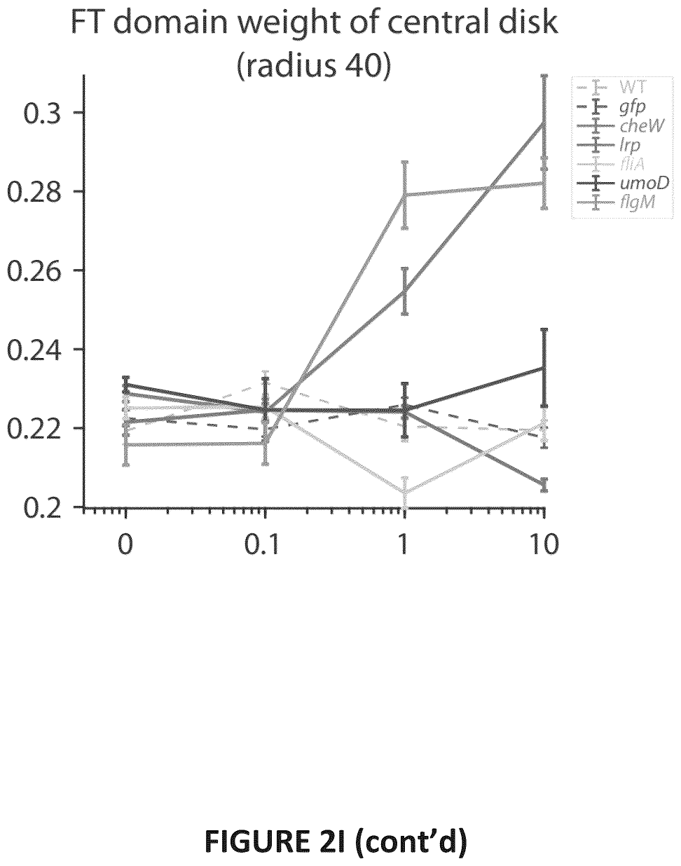

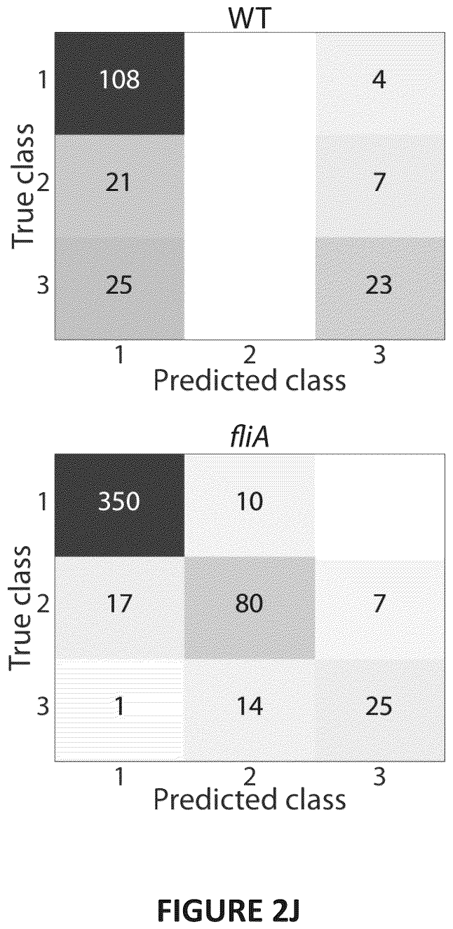

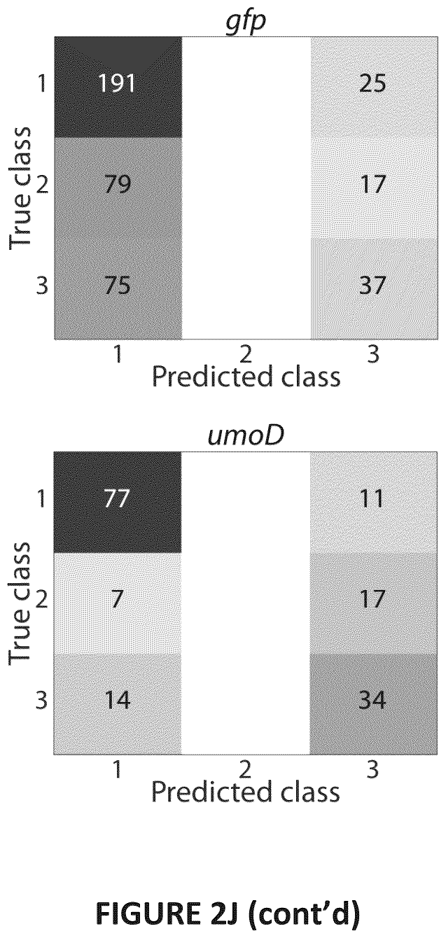

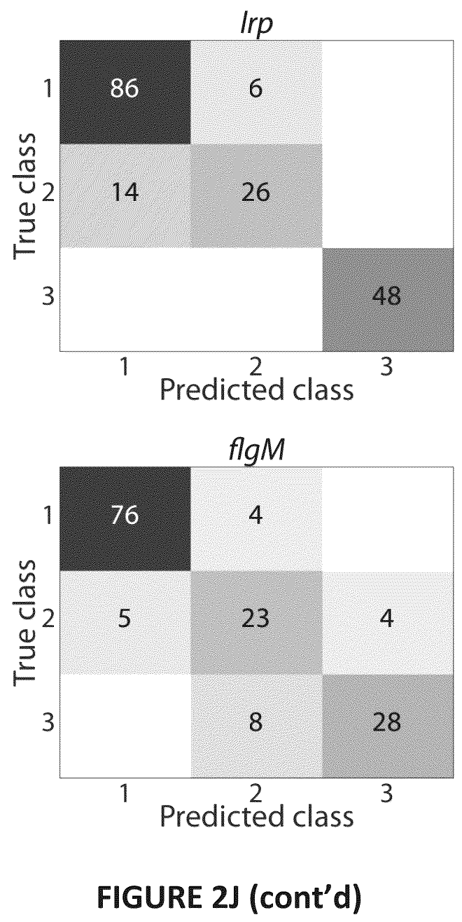

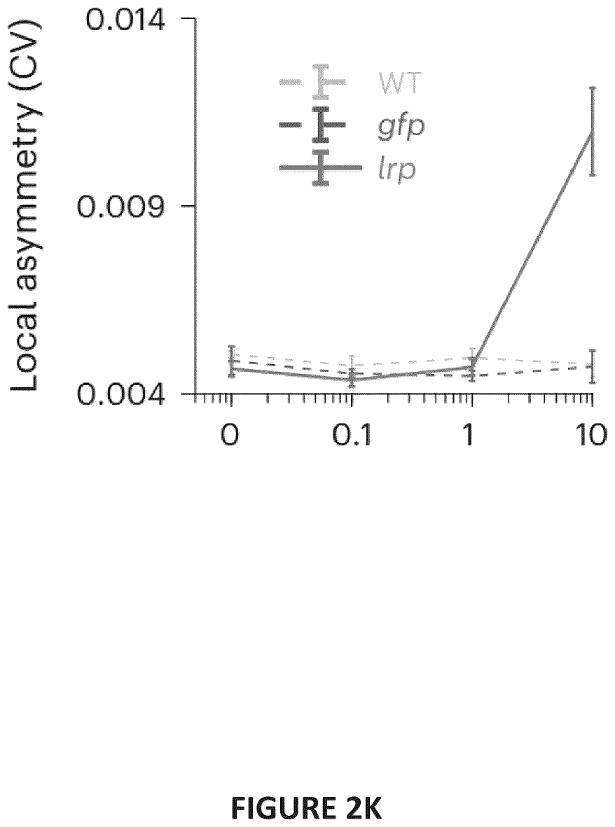

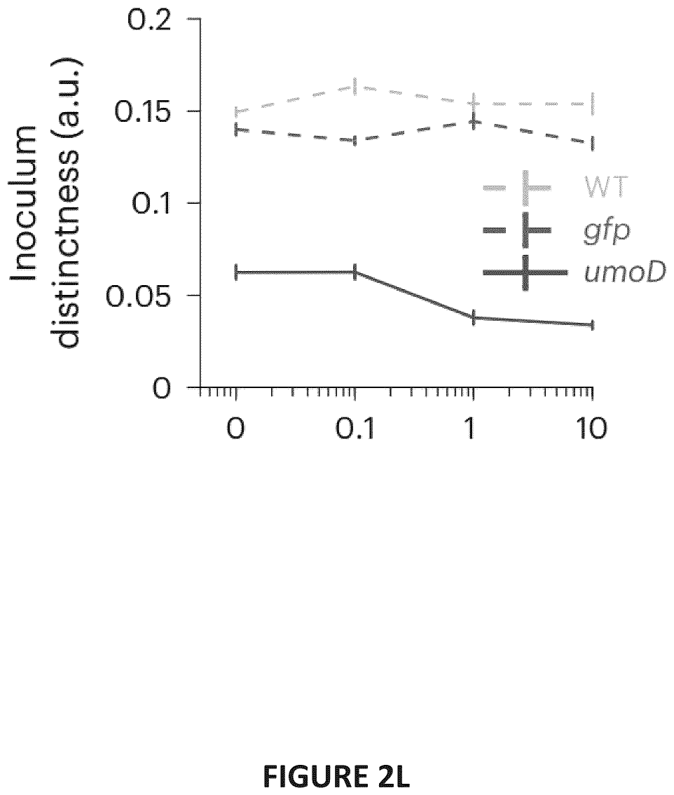

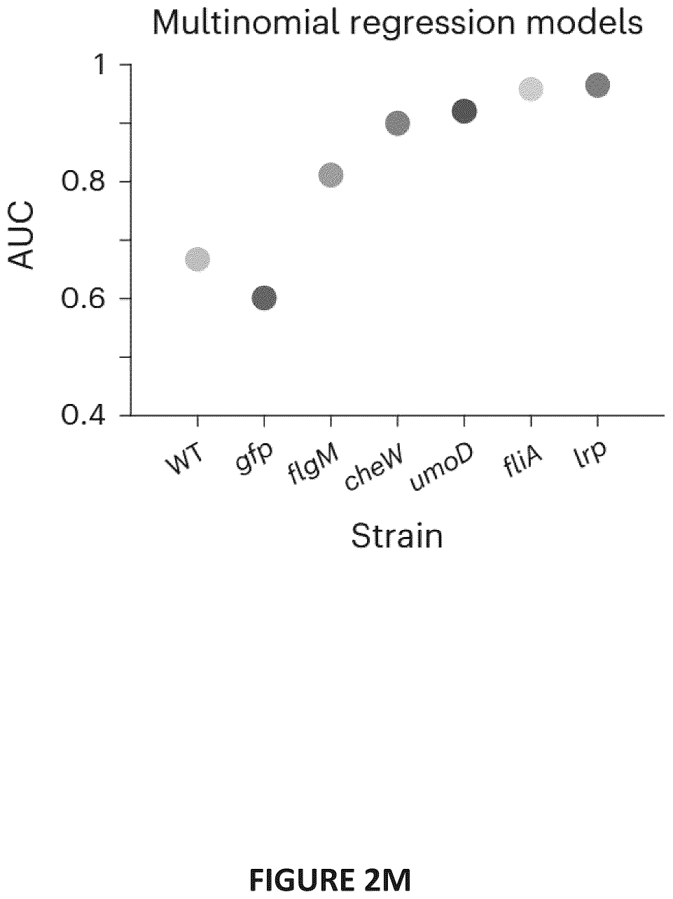

To build a system which could interpret multiple signals simultaneously, additional genes were sought that could modify distinct colony pattern features (FIG. 2A). Genes previously implicated in the swarm process were chosen, including fliA and flgM, which are involved in flagellar gene transcription; umoD, which controls the master regulator of swarming flhDC; and lrp, which affects general cellular processes in response to the presence of leucine. Overexpression of these genes via IPTG induction resulted in a variety of patterns formed, ranging from dense ruffled textures to “spikes” to indistinct ring boundaries (FIG. 2B). For comparison, a strain overexpressing flgA had no discernible pattern change with IPTG induction, confirming that the unique pattern changes were due to the overexpression of the swarm genes chosen (FIG. 2C). Scanning a range of IPTG concentrations showed graded changes in patterns (FIG. 2D). For example, with increased overexpression the lrp strain formed spikes in the inner rings, and at maximal induction each ring boundary was spiky. flgM overexpression caused colony radius to shrink, with few, small swarm rings beyond the initial inoculum visible at 24 hours. fliA overexpression caused the formation of more visible dots or “microcolonies” just within the boundaries of each ring with increased IPTG. As umoD overexpression increased, colonies became more symmetric, and ring boundaries and the inoculum edge became fainter. Taken together, these various qualitative characteristics suggested overexpression of certain swarm genes could indeed affect several pattern features that could be measured and quantified.

To quantitatively interpret these patterns, images were transformed to polar coordinates to enable ease of averaging across rows of the transformed images (FIG. 2E). Plotting the averaged profiles revealed distinct profiles for each strain (FIG. 2F). For example, overall colony density was higher with cheW overexpression than for the umoD strain. The spikes visible in the lrp pattern reduced the sharpness of the ring boundaries in the radially averaged profiles. The original images were also transformed to the Fourier domain to observe the frequencies present in each pattern (FIG. 2G). The periodic features in the patterns resulted in high visible intensities in certain areas of the Fourier transform images. For example, for the umoD strain, there was a higher magnitude in the high-frequency outer regions of the image, while for the other strains greater magnitude was at the central lower-frequency region.

The features of each strain that would allow for determination of the input IPTG concentration from endpoint images scanned after 24 hours of growth were subsequently identified. The first feature quantified was prevalence of low-frequency information, calculated as the sum of intensities within a disk-shaped region around the origin of the Fourier spectrum (similar to a low-pass filter) relative to the sum over the whole image. This measurement was found to increase with IPTG induction for the flgM and cheW strains, suggesting that at higher IPTG there was a greater magnitude of low-frequency information present in the image, corresponding to the visual observation of greater density of the central colony region and thinner, smaller rings in the patterns (FIGS. 2H-J). A second measure, the local coefficient of variation (CV) increased with increasing IPTG for the lrp strain, which could be observed visually in the spiked rings (FIG. 2K). Finally, the distinctness of the inoculum border, measured by the change in intensity over the border, was found to decrease with increasing IPTG for the umoD strain, particularly from 0.1 to 1 mM IPTG. (FIG. 2L). These measurements showed that overexpression of these genes could quantifiably affect the pattern in response to changes in IPTG.

To develop approaches for decoding information from the patterns, a dataset of images was generated for each strain grown at a range of inducer concentrations. Samples were binned into three classes, 0-0.09, 0.1-0.9, and 1-10 mM IPTG. Multinomial regression models were then fit to the data to see whether they could back-predict the input concentration class from an image sector's measurements, trying each parameter individually and every possible combination of the parameters as inputs into the models. The performance of such models can be evaluated using a multi-class area under the receiver-operating curve (AUC) metric, where the more accurate a model is for predicting true positives compared to false positives for each class, the closer the AUC will be to 1. The AUC of each strain's fitted model was evaluated on the input data (FIGS. 2I, 2J, 2M). For each strain, the combination of parameters which gave the highest AUC was selected for use in the final model. The chosen models for the experimental strains with cheW, fliA, lrp generally had AUC>0.9, showing that the models were well able to differentiate true positives in each IPTG class from false positives. The AUCs were 0.6 for the gfp control strain, just slightly above a random classifier (AUC=0.5), suggesting that its pattern parameters were not affected by increasing IPTG for control strains. The confusion matrices showed that the fitted models correctly classified a majority of the plates for each strain (FIGS. 2I and 2J). Thus, information about the environment encoded within the engineered strains' patterns can be decoded using combinations of relevant pattern features.

### Example 10

Dynamics of Engineered P. mirabilis Strains

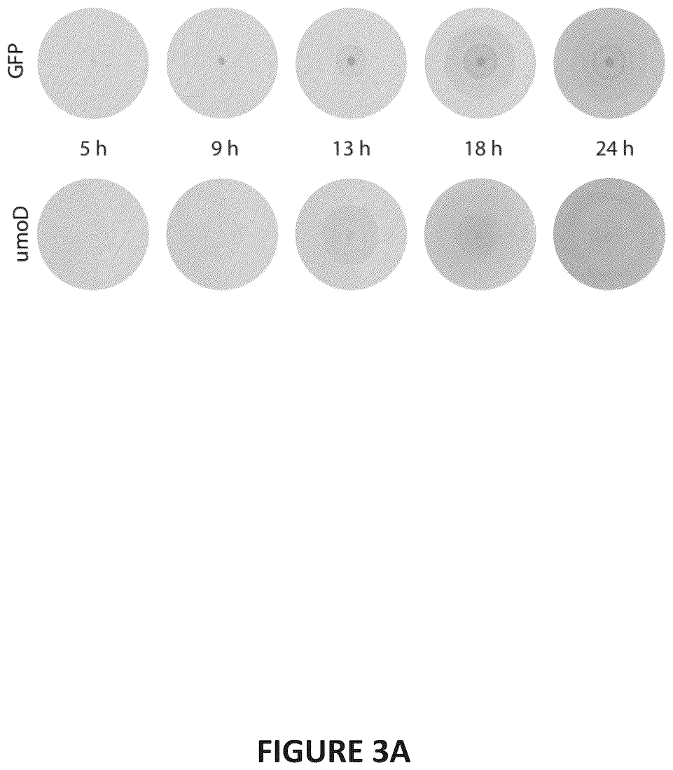

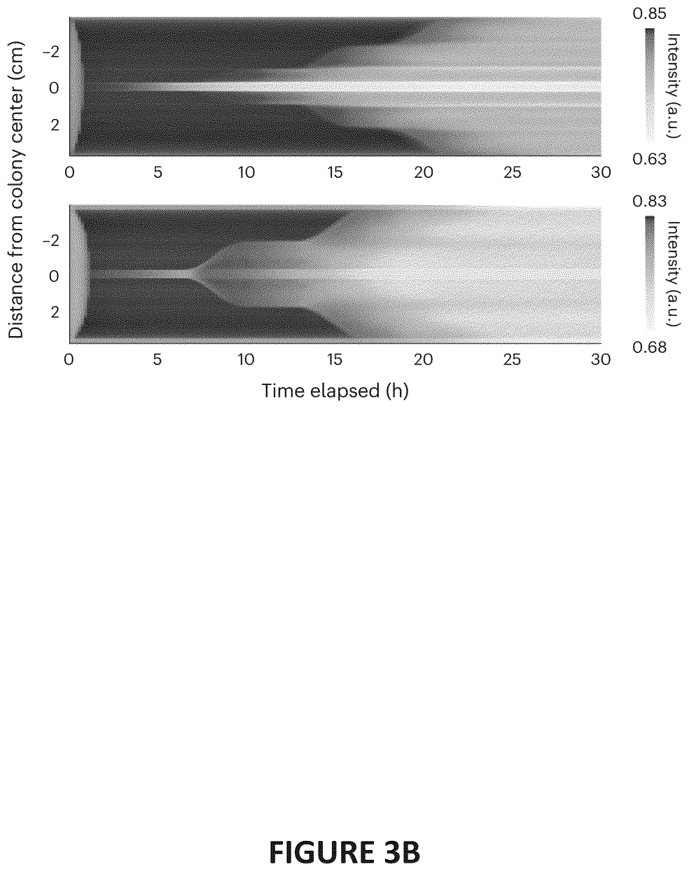

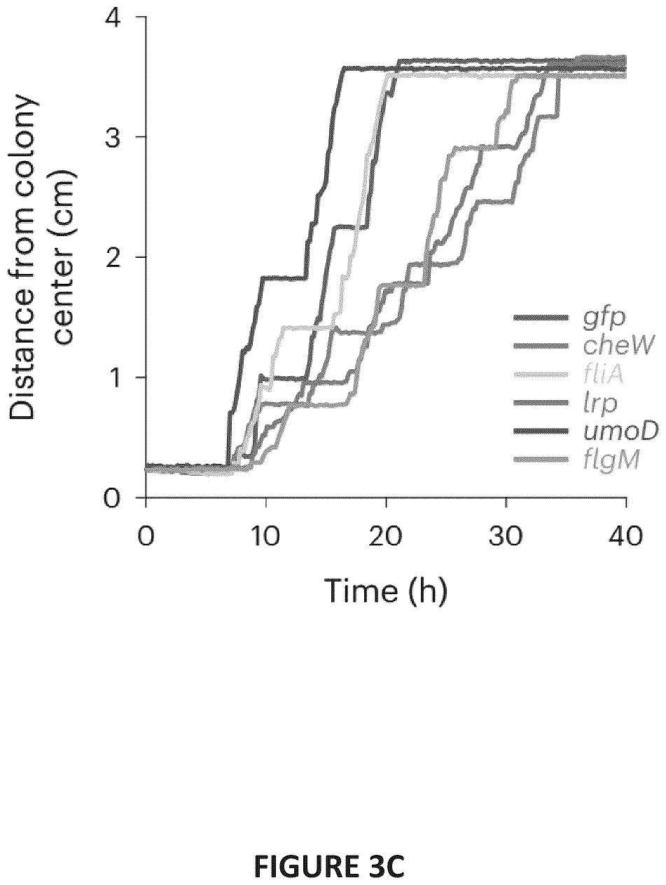

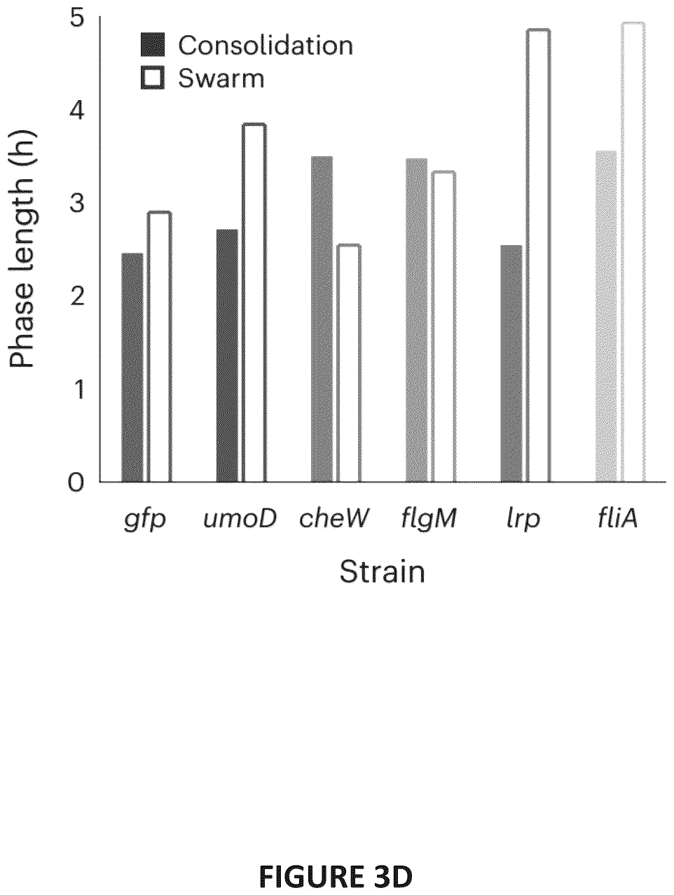

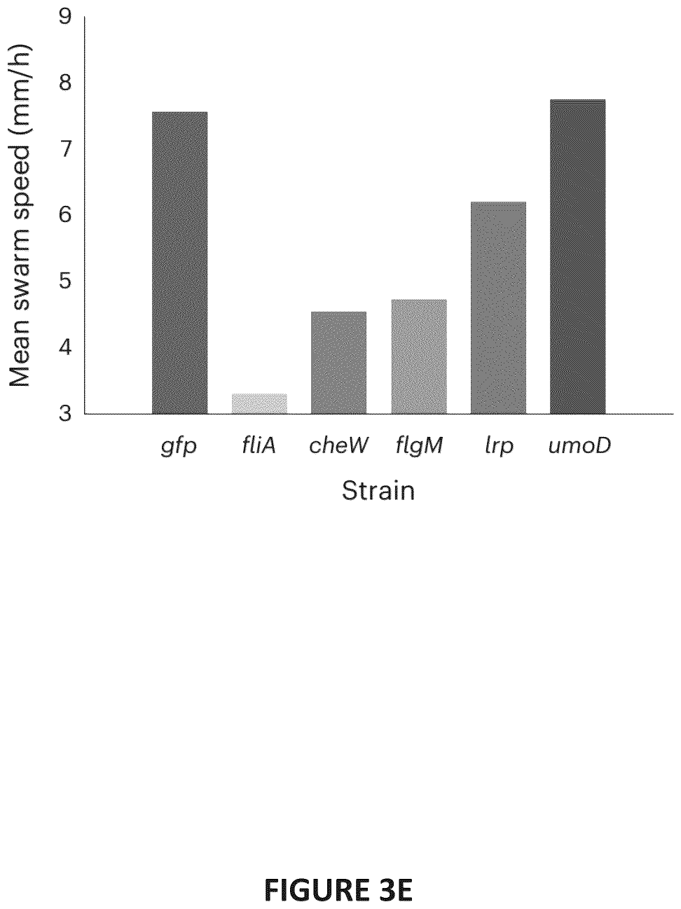

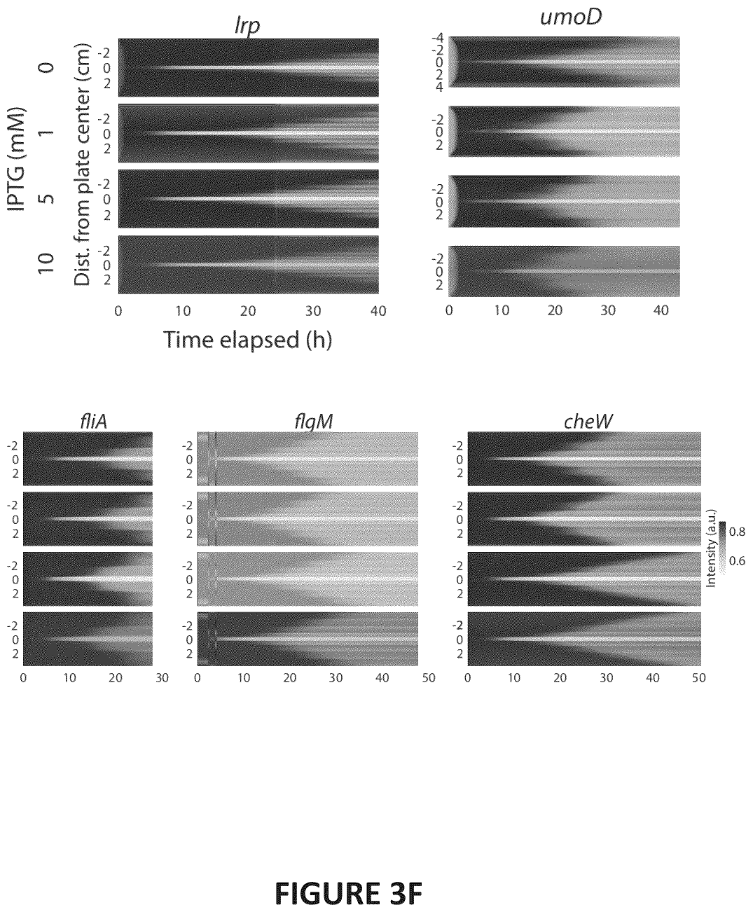

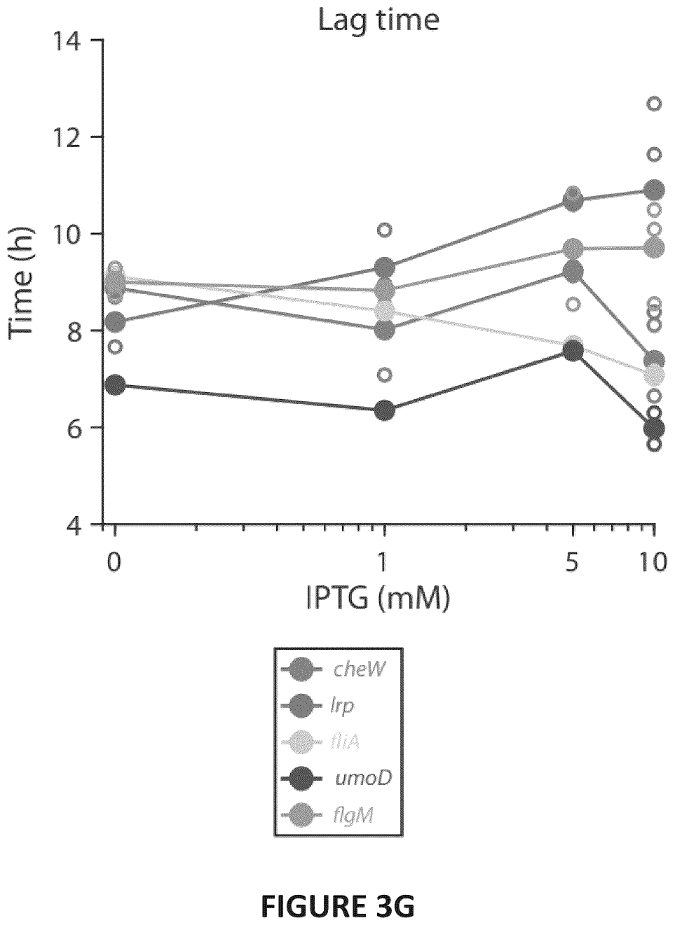

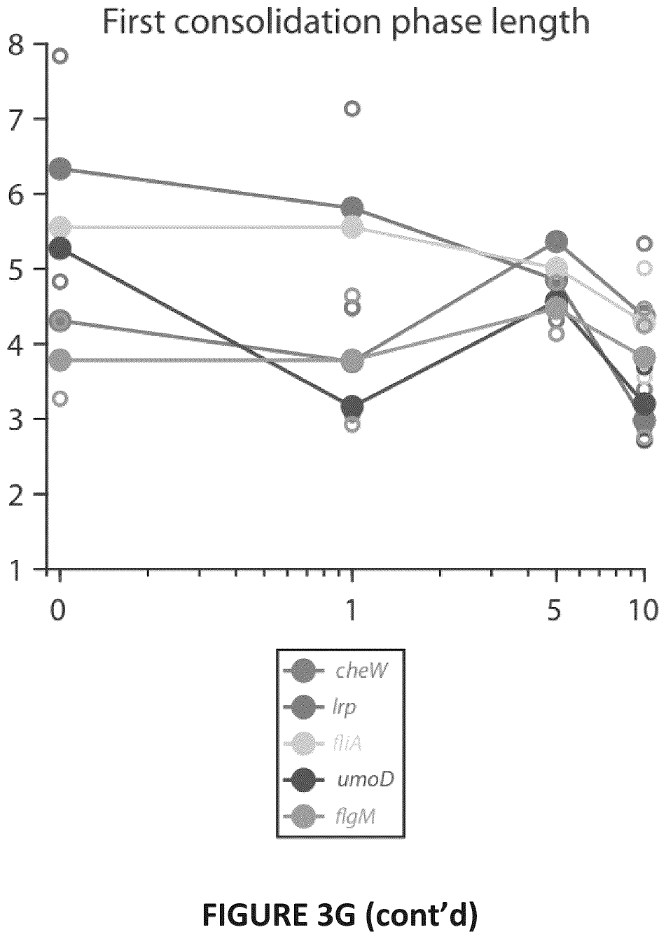

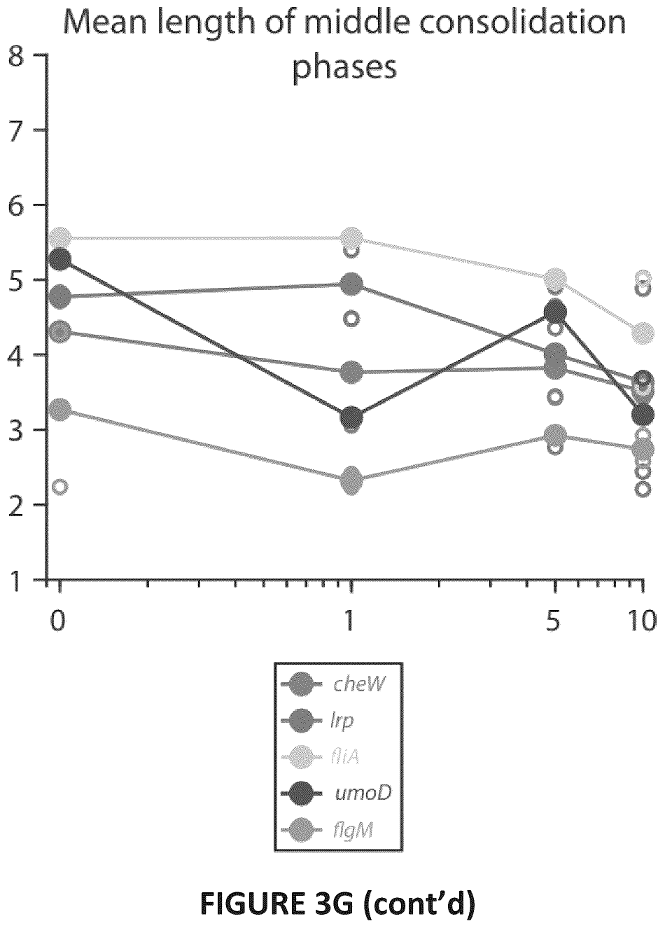

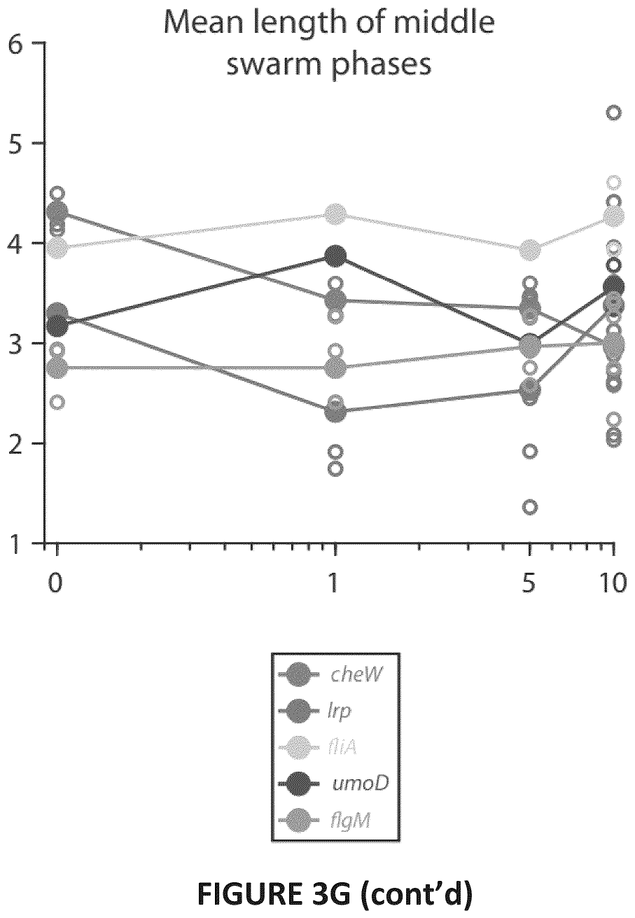

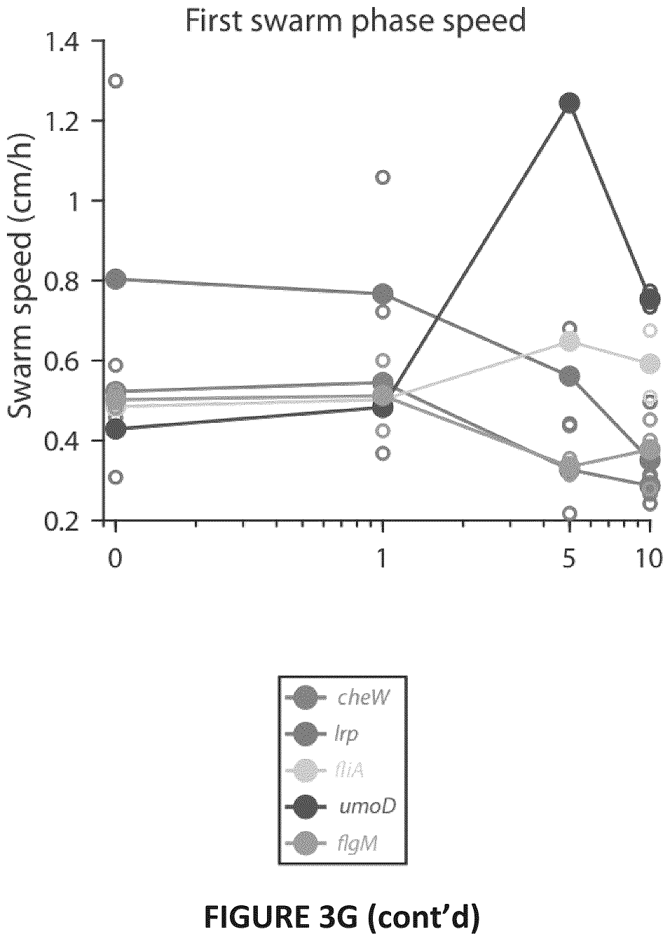

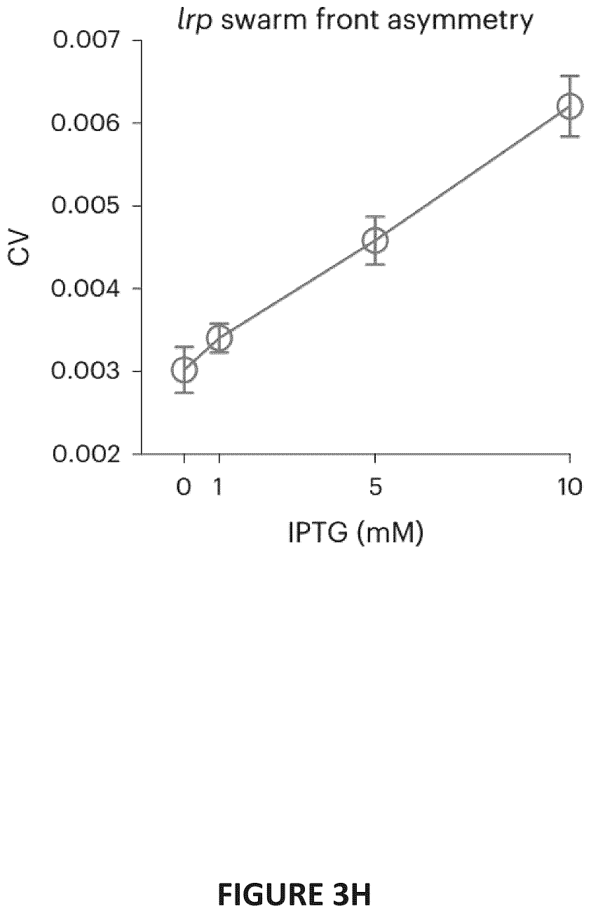

P. mirabilis swarming creates patterns not only in space, but also in time; this temporal regularity suggests the possibility of encoding information in both the endpoint patterns and their dynamic growth phases. The dynamics of the engineered strains were thus investigated using time-lapse imaging of colony growth (FIG. 3A). For each strain, a time-lapse was captured with maximal IPTG induction at 25° C.; images were taken every 10 minutes (FIG. 3A). The individual time-lapse images were then averaged in polar coordinates and visualized via heatmaps (FIG. 3B). The location of the colony front at each timepoint was identified and trajectories were obtained with high spatio-temporal resolution (FIG. 3C). The colony growth trajectories showed that each of the engineered strains maintained the classic alternation in phases, but with changes in aspects such as initial lag time and length of the phases compared to the control gfp strain. Lag, consolidation, and swarm phases from each of these trajectories were then measured (FIG. 3D), as well as the distance swarmed during each swarm phase, allowing for calculation of swarm speed (FIG. 3E).

Subsequently, individual time-lapses of each strain grown at a range of IPTG concentrations were recorded (FIG. 3F). Induced overexpression of the swarming genes affected each of these parameters; for example, the first consolidation phase of the lrp, fliA, and umoD strains was observed to be shorter at 10 mM IPTG than at 0 mM IPTG. Meanwhile, the mean speed during the first swarm phase was reduced with IPTG induction for the cheW and lrp strains, but increased for the umoD strain (FIG. 3G). More complex dynamic parameters also encoded information; for example, the asymmetry of the colony front during swarming phases increased with IPTG for the lrp strain (FIG. 3H). The strain's lag time, a parameter which can be measured much sooner than endpoint pattern features, also increased with IPTG (FIG. 3G). These results suggest that dynamic parameters can also be used to encode and decode information from these spatiotemporal patterns.

### Example 11

### Multiplexed Recording Using a Dual-Input Strain

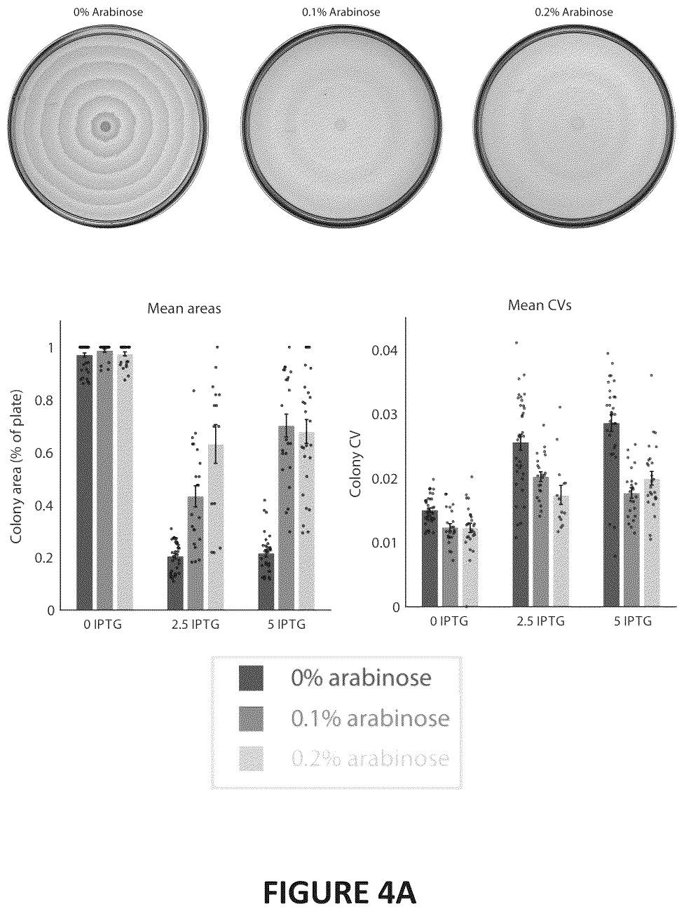

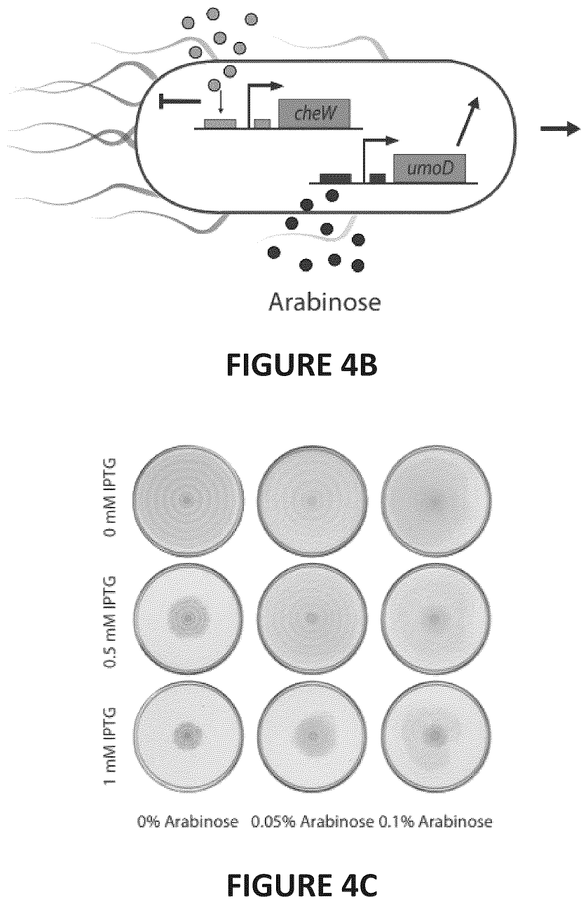

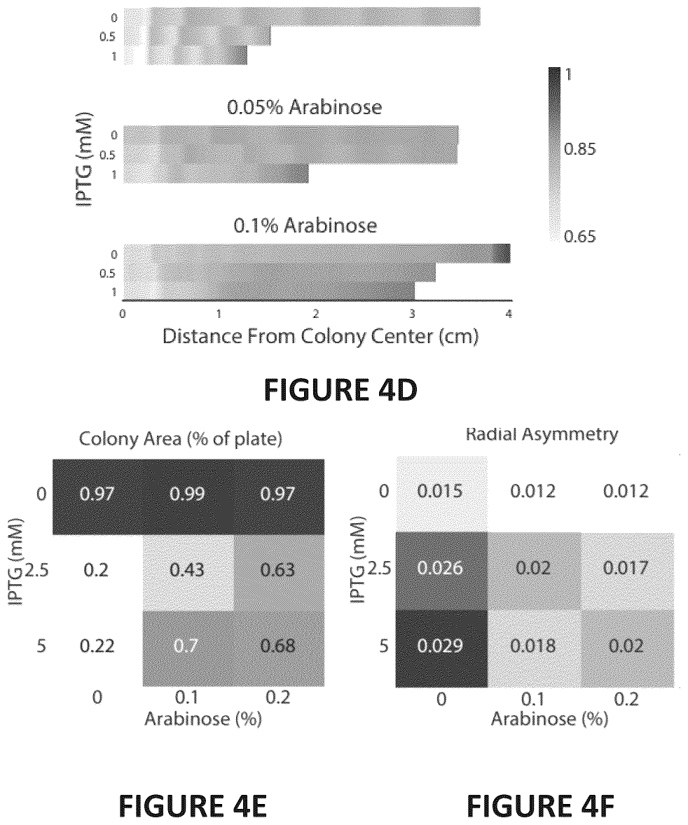

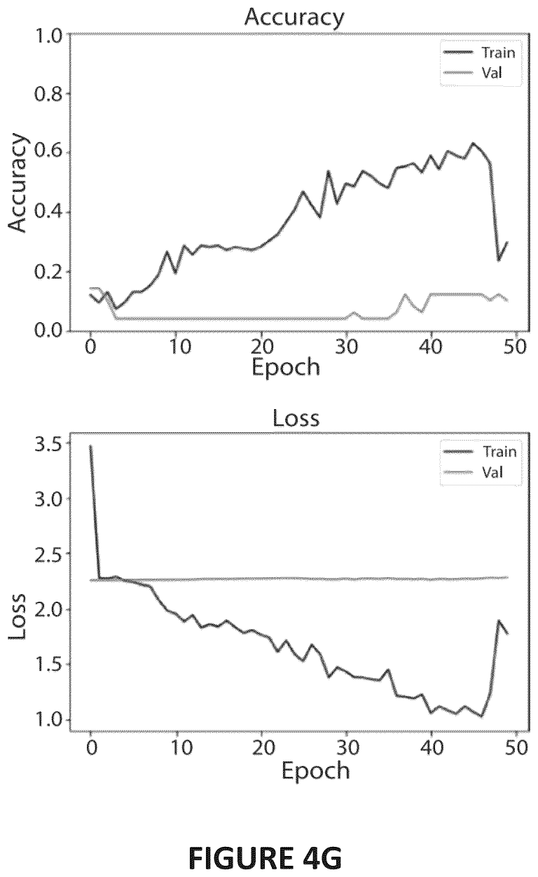

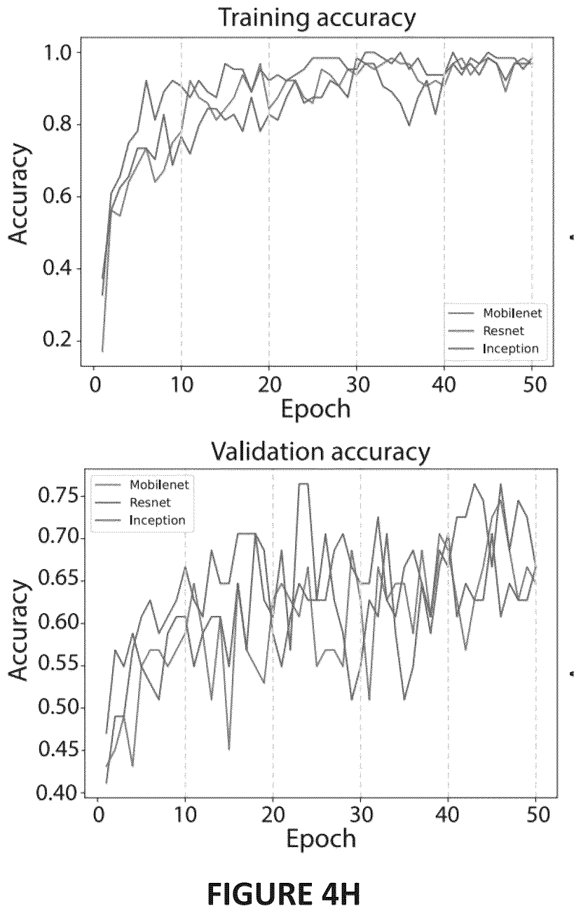

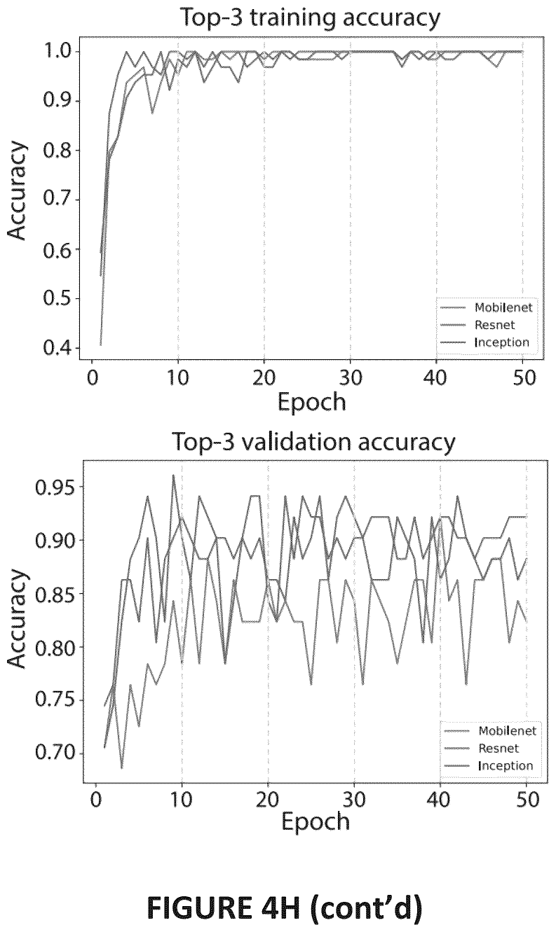

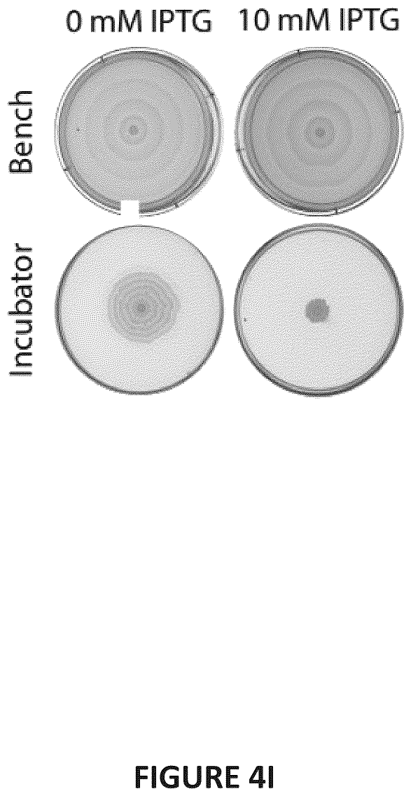

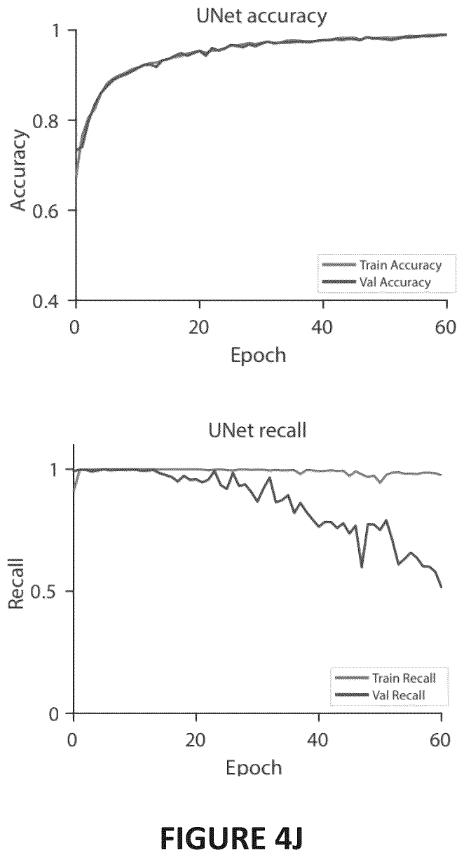

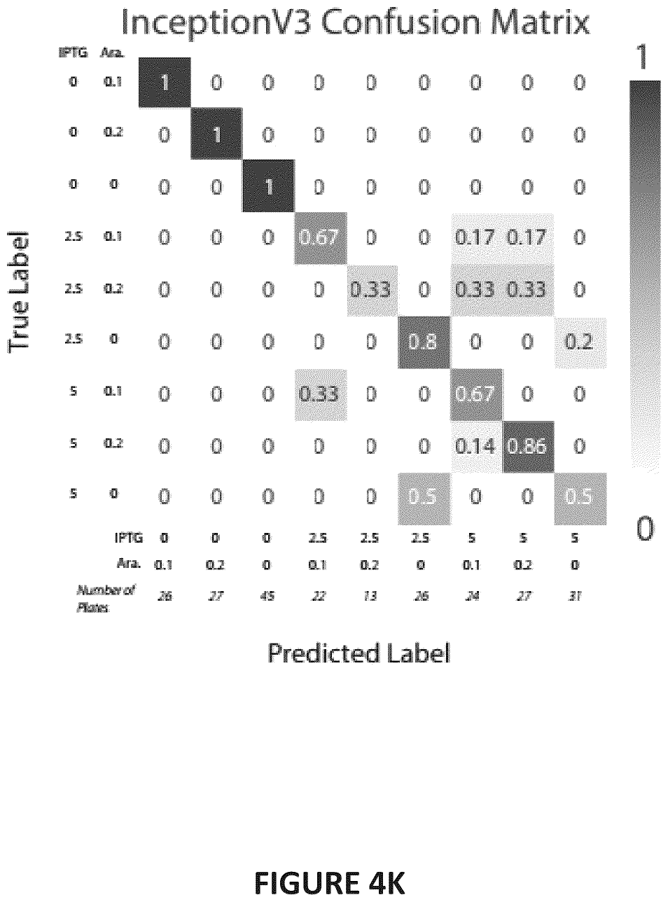

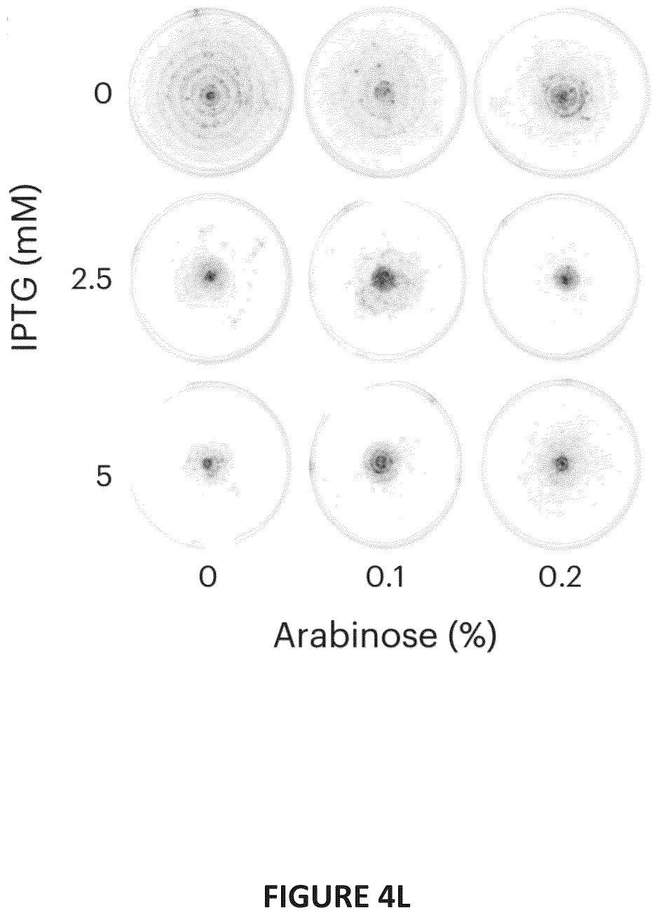

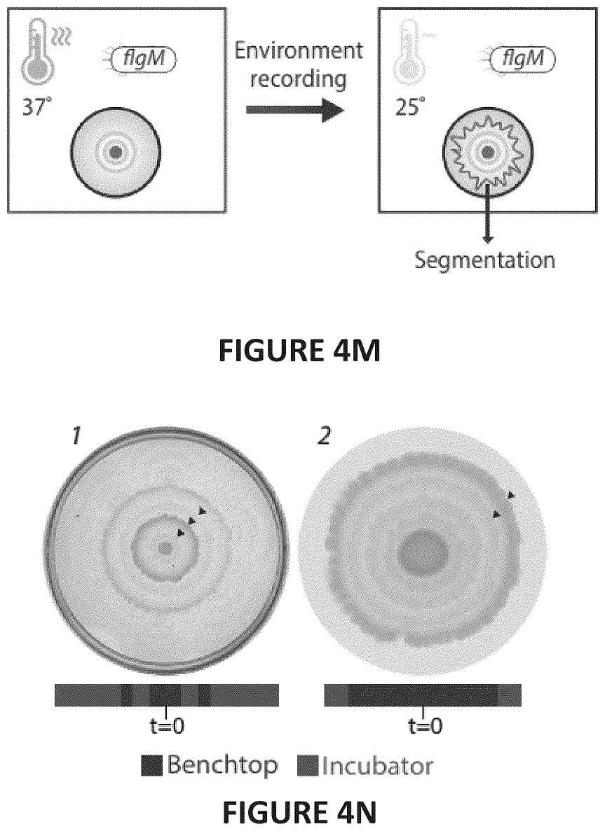

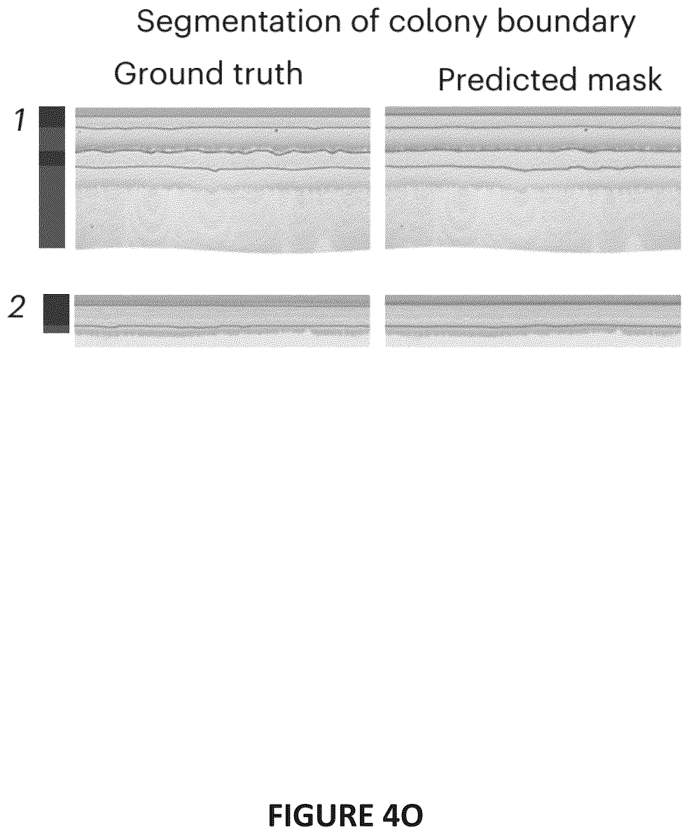

In order to build a strain which could provide information about two inputs simultaneously, a second swarming-related gene was induced with the pBAD operon and promoter, transcribed in the presence of the molecule arabinose (FIG. 4A). This combination strain, with cheW overexpression induced by pLac, and umoD overexpression induced by pBAD, was designed with the understanding that swarming-related genes have interdependent effects and these two genes could robustly change distinct pattern features on their own (FIGS. 4A and B). Initial characterization of this strain demonstrated that its swarm patterns indeed distinctly reflected the presence or absence of each input (FIG. 4C). Representative radially-averaged profiles were visualized as heatmaps for comparison (FIG. 4D). The plates imaged followed a characteristic pattern at most of the conditions. Increasing IPTG from 0 to 1 mM, inducing cheW overexpression, resulted in a visible decrease in 24-hour colony radius, ring width, and colony symmetry, as seen previously in the single input strain. Meanwhile, increasing arabinose from 0 to 0.1% resulted in a highly symmetric pattern with initially semi-distinct, narrow rings giving way to the indistinct wide rings more characteristic of the single-input umoD pattern. The combination of IPTG and arabinose presence resulted in a similar pattern, with narrower inner rings giving way to wider outer rings, slightly smaller colonies, and not-quite-symmetric colonies.

To characterize the cheW and umoD combination patterns in more detail, a dataset of plate images at IPTG concentrations of 0, 2.5, and 5 mM combined with arabinose at 0%, 0.1%, and 0.2% was created. Higher concentrations of the inducers were used in order to generate greater differences in the pattern, with the aim of ease of decoding. The average percent of the plate covered by the colony at each condition decreased with increasing IPTG and increased with the addition of arabinose (FIG. 4E). However, increase of arabinose from 0.1% to 0.2% had little noticeable effect on the colony area except at 2.5 mM IPTG (FIG. 4A). Similarly, average radial CV as a measure of colony asymmetry increased with the overexpression of cheW, but decreased with the addition of arabinose inducing umoD overexpression (FIGS. 4A and F).

As done previously for the single-input strains, a set of standard measurements was then taken on each image in the dataset, and a 9-class multinomial regression model was fit on the output. See Table 1 below.

TABLE 1
Multinomial regression model performance
on dual input strain measurements

| | | | | | | | | | | |
|---|---|---|---|---|---|---|---|---|---|---|
| True Class | 1 | 47 | 15 | | | | | | | |
| | 2 | 23 | | 3 | 1 | | | | | |
| | 3 | 18 | 3 | 1 | 9 | | | | | |
| | 4 | 7 | 45 | 8 | | | | | | |
| | 5 | 4 | 16 | 6 | | | | | | |
| | 6 | 1 | 15 | 4 | 1 | | | | | |
| | 7 | 2 | 28 | 9 | | | | | | |
| | 8 | 1 | 1 | 15 | 6 | | | | | |
| | 9 | 4 | 8 | 16 | 2 | | | | | |
| | | 1 | 2 | 3 | 4 | 5 | 6 | 7 | 8 | 9 |

Predicted Class

| | Weights |
|---|---|
| Col. Radius (Pixels) | −0.0223 |
| Col. Radius (cm) | 3.7914 |
| Reached Edge of Plate | 2.5727 |
| Mean Colony Intensity | −11.2744 |
| Mean Sliding CV | −48.8359 |
| Mean Inoculum Intensity | −18.9936 |
| Max Inoculum Intensity | 18.8092 |

The mnrfit function was used to obtain a best fit model for classifying sets of measurements into nine bins: 1: 0-1 mM IPTG, 0-0.1% arabinose; 2: 1-5 mM IPTG, 0-0.1% arabinose; 3: 5-10 mM IPTG, 0-0.1% arabinose; 4: 0-1 mM IPTG, 0.1-0.19% arabinose; 5: 1-5 mM IPTG, 0.1-0.19% arabinose; 6: 5-10 mM IPTG, 0.1-0.19% arabinose; 7: 0-1 mM IPTG, 0.2% arabinose; 8: 1-5 mM IPTG, 0.2% arabinose; 9: 5-10 mM IPTG, 0.2% arabinose. The confusion matrix was then plotted; numbers on the matrix represent numbers of plates. The weights on each variable used for the best fit model are shown below the table.

The model performed poorly, predicting all images as 0% arabinose, and the maximum AUC achieved was only 0.72. This result suggested that the two-input strain's patterns, involving interdependent swarm genes, was too complex for the previous regression-based decoding method. However, the ease of distinguishing the patterns by human eye suggested that the application of deep learning methods for image classification could prove useful for decoding the patterns. In particular, deep convolutional neural networks (CNNs) have clear applicability and have not yet been used to characterize macroscale bacterial colony patterns. CNNs can learn to extract salient features from bacterial images and classify patterns to predict the image class.

Accordingly, a simple CNN was created from scratch using the data and fine-tuning deep networks pre-trained on ImageNet data, including ResNet and Inception architectures (FIGS. 4G-4J). The fine-tuned InceptionV3 model was able to successfully classify the majority of our the images; it achieved a mean test accuracy of 0.83 and top3-accuracy of 0.98, and an AUC=0.96 for the micro-averaged ROC curve, a noticeable improvement from the multinomial regression model. It was observed that intermediate concentrations of IPTG and arabinose reduced the model's accuracy due to some bimodality in pattern formation (FIG. 4K). Visualizing the pixel attributions of the model indicated the inoculum and inner rings had a large impact on the predictions, suggesting that these areas of the pattern were most affected by the overexpression of the different swarm genes (FIG. 4L). Since the innermost portion of the colony was most critical to pattern prediction, pattern decoding may be possible after just a few hours of growth, rather than needing to wait 24 hours until the full plate is covered.

Since the classification approach requires all potential concentrations to have been acquired in the training dataset, a regression-output approach was also explored to predict intermediate values that would provide more utility for future applications. The previous dataset was combined with images acquired at additional concentrations of IPTG and arabinose. When the EfficientNetB2 architecture, pre-trained on ImageNet, was trained on a subset of this new dataset, it achieved mean squared errors of less than 0.1 on held-out validation and test subsets and was able to reasonably predict IPTG and arabinose over the range of concentrations seen. Overall, these results suggest that this system can be used to encode and decode multiple inputs, and that the use of deep networks along with transfer learning will enable decoding of complex pattern feature changes.

### Example 12

Multi-Condition Pattern Segmentation and Information Decoding with Deep Learning

Whether an engineered strain could record changes in the environment taking place during pattern formation and how these changes could be decoded from the endpoint pattern, similar to the analysis of rings in a tree, was investigated. This experiment utilized the flgM strain, which was observed to form two strikingly different patterns in the presence of 10 mM IPTG in the incubator vs on the benchtop: a swarming-inhibited, ruffled, dense pattern in the incubator at 37° C., and a wide-ring, symmetric, less dense pattern on the benchtop at ˜25° C. (FIG. 4I). After inoculation, plates were first placed in one condition; after some time, plates were switched to a second condition, and certain plates were switched a third time before the endpoint scans were captured (FIG. 4M). Plates were scanned before each switch, creating a dataset of 21 images. Representative pattern images are shown in FIG. 4N. This shift in environmental conditions resulted in the formation of rings alternating between indistinct, radially symmetric, wider rings and dense, asymmetrical, narrow rings, visible as bands on the polar-transformed images. In general, denser regions corresponded to incubator growth, while fainter regions with wider rings corresponded to benchtop growth.

Towards decoding these alternating ring patterns, the dataset was manually annotated, creating ground truth masks of the boundaries marking the shift in the pattern corresponding to a shift in the environment. A U-Net model was then trained on the data with a VGG-11 encoder pretrained on ImageNet to predict these boundaries given an input pattern image. This model achieved above 95% training and validation accuracy and above 90% recall within the first 25 epochs of training (FIG. 4J). Application of the trained model to previously unseen images resulted in specific prediction of boundaries matching the ground truth, and noticeably did not simply highlight all ring boundaries. Taken together, these data demonstrated that this approach could be used to decode information about changing environment from the engineered strains' patterns (FIG. 4O).

### Example 13

Engineering P. mirabilis to Detect Copper

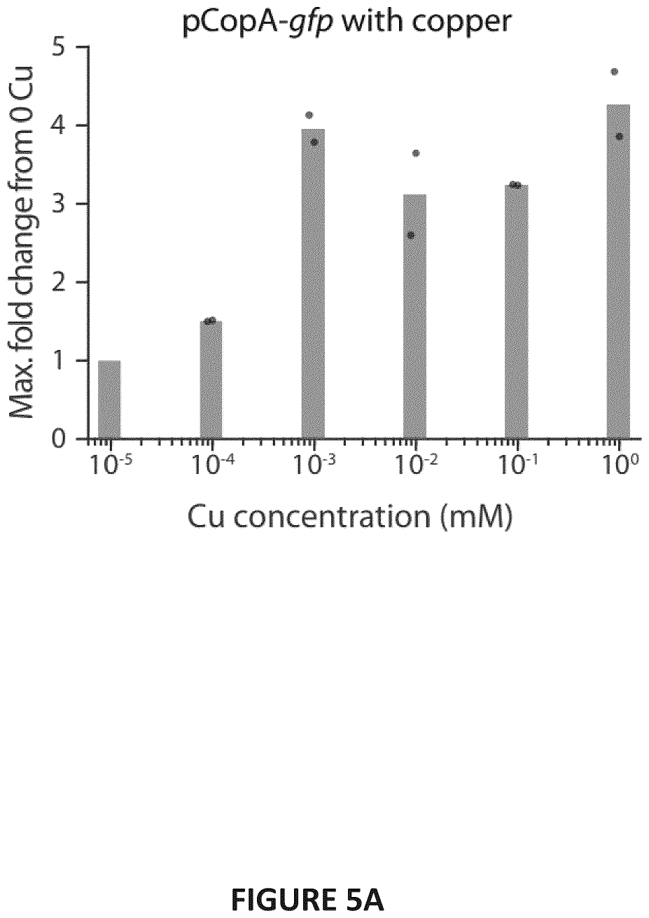

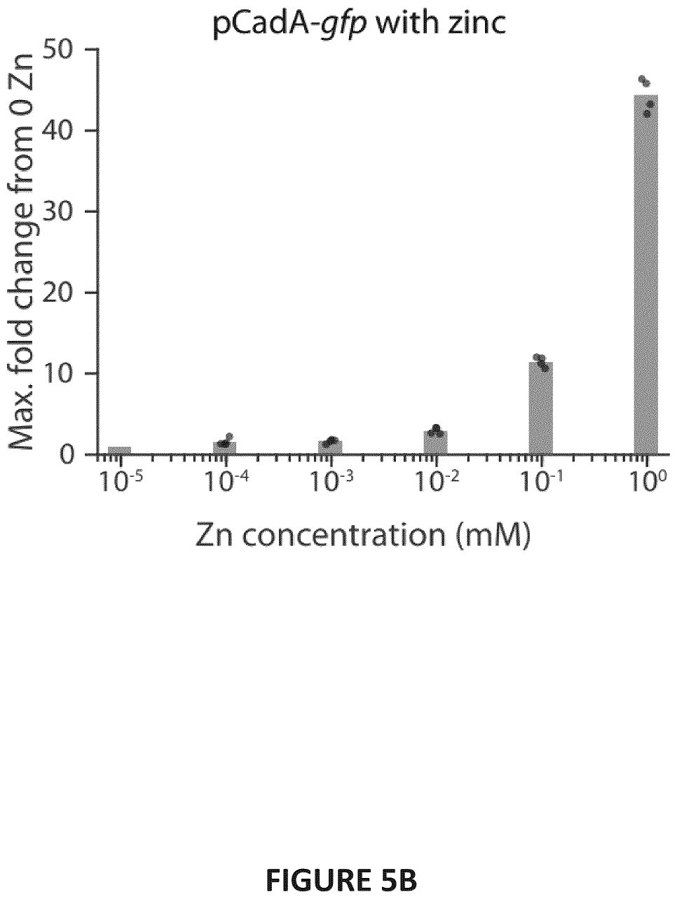

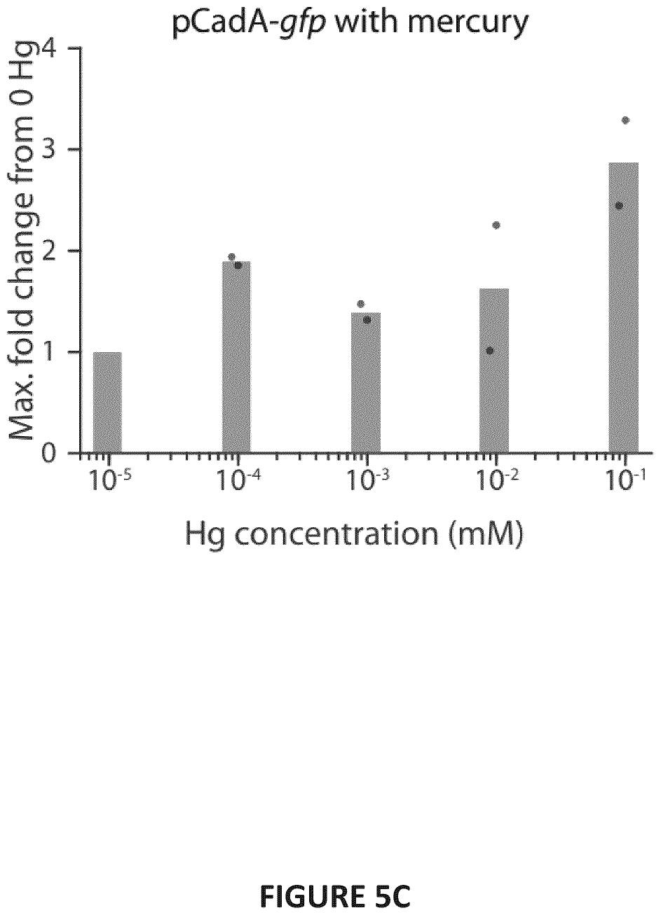

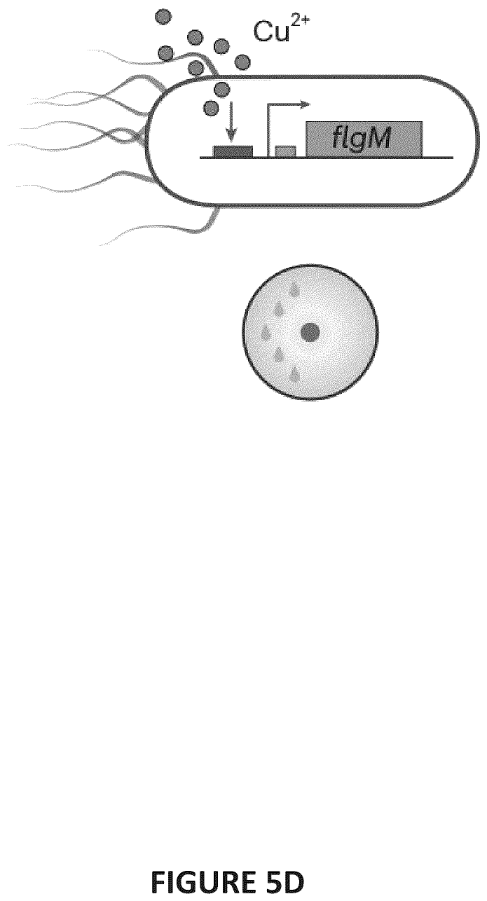

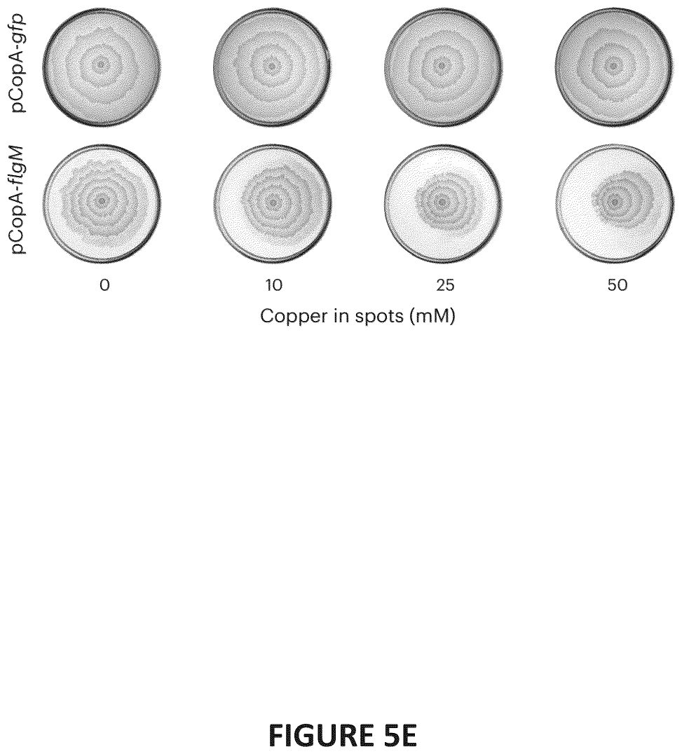

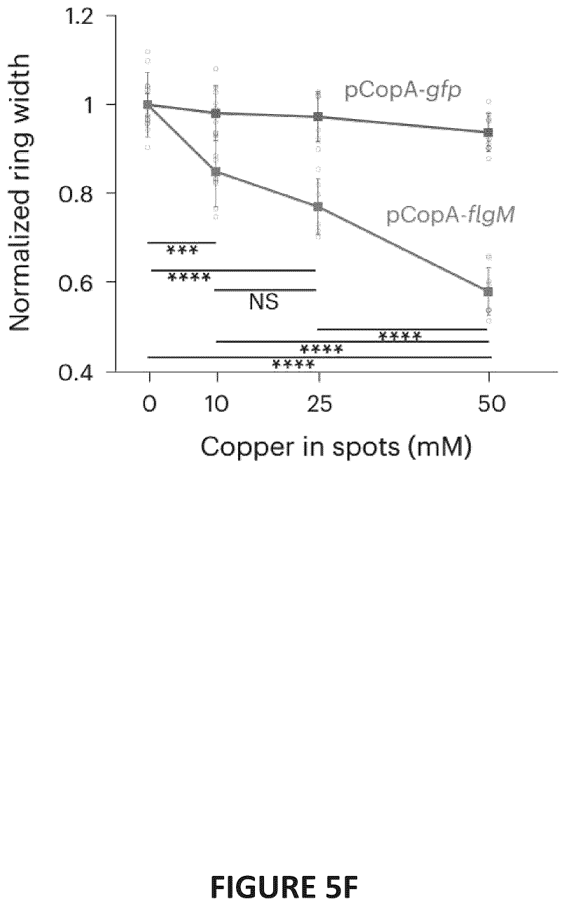

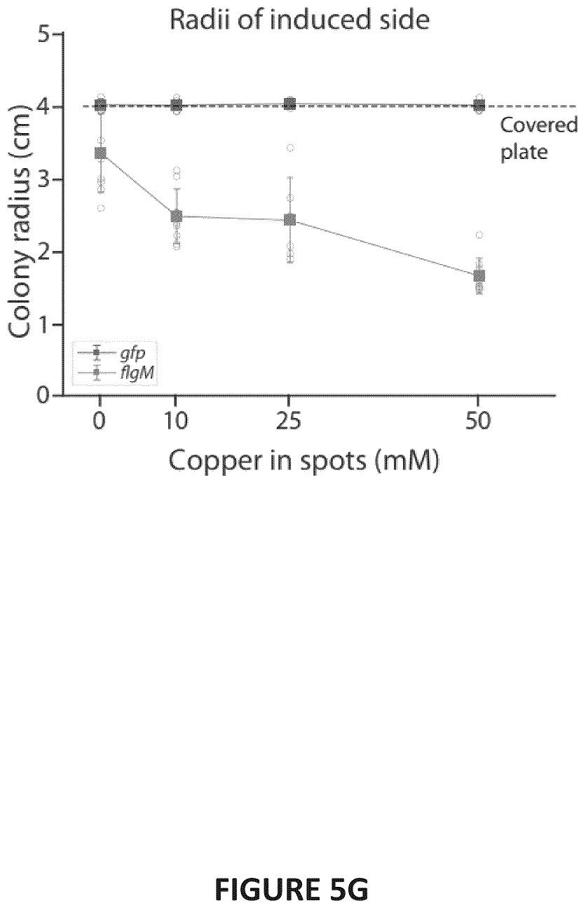

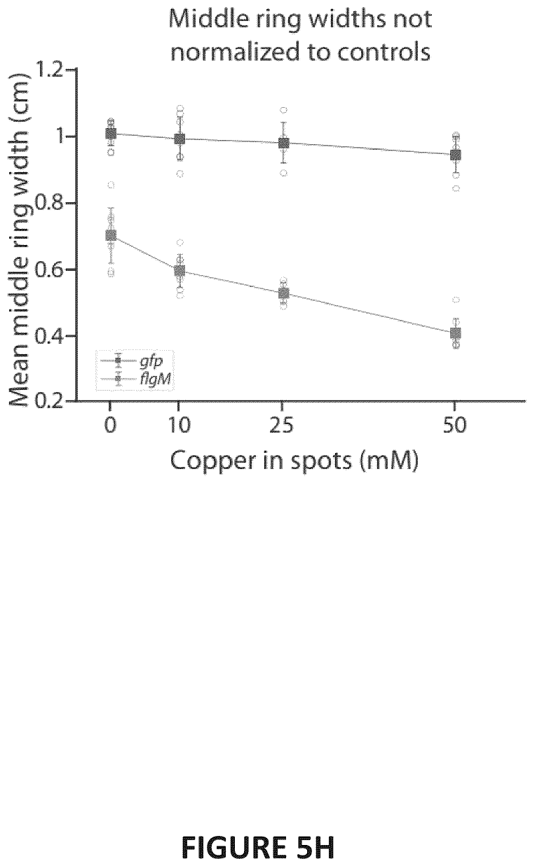

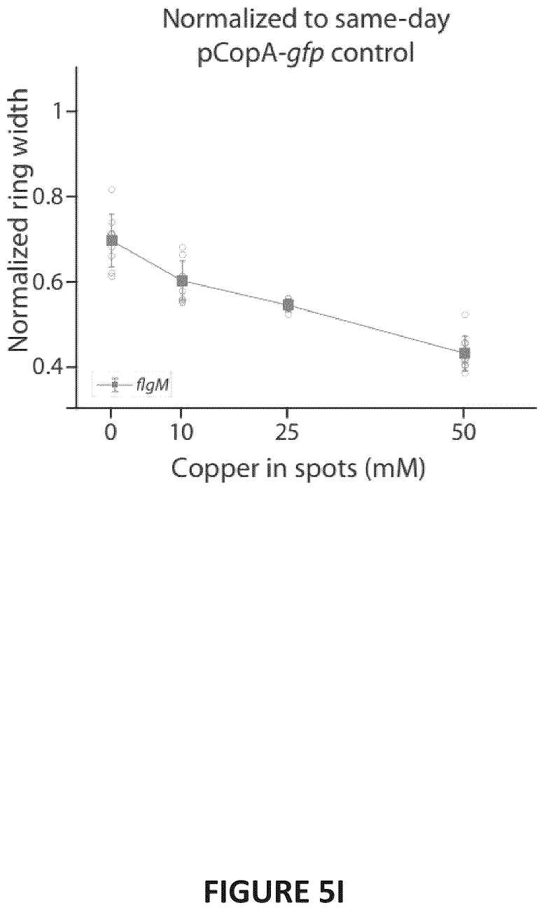

Subsequent experiments were conducted to determine whether the engineered strains could be used to detect inputs beyond IPTG and arabinose. Heavy metal detection was chosen given the innate resistance of P. mirabilis to certain heavy metals. Intergenic regions, containing putative promoters, were cloned before the native P. mirabilis 7002 copA (copper-exporting P-type ATPase) and cadA (heavy metal-translocating P-type ATP-ase, for zinc, cadmium, and mercury) onto the previously-used pLac-GFP plasmid, replacing the pLac promoter region. A plate reader experiment for GFP expression demonstrated that the copA promoter responded to the presence of increasing concentrations of copper from 0.001 to 1 mM, and the cadA promoter responded to zinc (0.001 to 1 mM) and mercury (0.0001 to 0.1 mM), by increasing GFP expression during log phase growth (FIGS. 5A-C). The GFP gene was then replaced with flgM and the strain's ability to read-out copper presence in spots of water was explored, using a modified protocol involving spotting samples on top of the agar for greater applicability (FIG. 5D). The pCopA-flgM strain visually recorded the presence of up to 50 mM copper in the water spots, with a graded change from 0 to 50 mM in the ring widths and colony radii at the spot locations (FIGS. 5E-5I).

While this invention has been disclosed with reference to particular embodiments, it is apparent that other embodiments and variations of the inventions disclosed herein can be devised by others skilled in the art without departing from the true spirit and scope thereof. The appended claims include all such embodiments and equivalent variations.

What is claimed is:

1. A method of detecting a spatiotemporal change in an environmental parameter, comprising:
(a) applying a plurality of a transgenic bacterium comprising at least one inducible promoter to a substrate, wherein the inducible promoter controls at least one gene related to swarming motility;
(b) detecting a difference between an observed swarming pattern of the plurality of the transgenic bacterium and an expected swarming pattern of the plurality of the transgenic bacterium on the substrate, wherein the difference is due to a spatiotemporal change in the environmental parameter.

2. The method of claim 1, wherein the transgenic bacterium is selected from the group consisting of P. mirabilis, P. aeruginosa, Paenibacillus vortex, B. subtilis str. 3610, and P. aeruginosa PA14.

3. The method of claim 1, wherein the at least one inducible promoter is induced by an agent selected from the group consisting of isopropyl ß-D-1-thiogalactopyranoside (IPTG) and arabinose.

4. The method of claim 1, wherein the at least one gene is selected from the group consisting of cheW, fliA, flgM, umoD, and lrp.

5. The method of claim 1, wherein the spatiotemporal change can be detected beginning from about six hours to about twenty four hours after applying the plurality of the transgenic bacterium to the substrate.

6. A method for performing macroscopic analysis of a plurality of bacterium, the method comprising:
(a) receiving at least one image of the plurality of a transgenic bacterium configured in a macroscopic orientation; and
(b) recognizing, within the image, a visual pattern of the plurality of bacterium and generating a mask that approximates the visual pattern of the plurality of a transgenic bacterium using one or more trained machine learning models.

7. The method of claim 6, wherein the bacterium is selected from the group consisting of P. mirabilis, P. aeruginosa, Paenibacillus vortex, B. subtilis str. 3610, and P. aeruginosa PA14.

8. The method of claim 6, wherein the visual pattern comprises at least one ring.

9. The method of claim 8, wherein the mask is generated by combining outputs from a first machine learning model and a second machine learning model.

10. The method of claim 9, wherein the first machine learning model and the second machine learning model comprise convolutional neural networks.

11. The method of claim 9, wherein the first machine learning model is configured to generate a mask that approximates a first portion of the visual pattern and the second machine learning model is configured to generate a mask that approximates a second portion of the visual pattern.

12. The method of claim 9, wherein the at least one ring comprises an outer ring and a plurality of inner rings, and the first machine learning model is configured to detect the outer ring and generate a mask that approximates the outer ring, and the second machine learning model is configured to detect the plurality of inner rings and generate a mask that approximates the inner rings.

13. The method of claim 9, wherein the first machine learning model is configured to detect the at least one ring by segmenting the image into multiple patches and distinguishing the outer ring from background in each patch.

14. A system for performing macroscopic analysis of a plurality of bacterium, the system comprising:
(a) a processor; and
(b) a memory storing instructions for execution by the processor, the instructions configuring the processor to:
(i) receive at least one image of a plurality of bacterium configured in a macroscopic orientation; and
(ii) recognize, within the image, a visual pattern of the plurality of bacterium and generate a mask that approximates the visual pattern of the plurality of bacterium using one or more trained machine learning models.

15. The system of claim 14, wherein the bacterium is selected from the group consisting of P. mirabilis, P. aeruginosa, Paenibacillus vortex, B. subtilis str. 3610, and P. aeruginosa PA14.

16. The system of claim 14, wherein the mask is generated by combining outputs from a first machine learning model and a second machine learning model.

17. The system of claim 16, wherein the first machine learning model and the second machine learning model comprise convolutional neural networks.

18. The system of claim 16, wherein the first machine learning model is configured to generate a mask that approximates a first portion of the visual pattern and the second machine learning model is configured to generate a mask that approximates a second portion of the visual pattern.

19. The system of claim 16, wherein the visual pattern comprises an outer ring and a plurality of inner rings, and the first machine learning model is configured to detect the outer ring and generate a mask that approximates the outer ring, and the second machine learning model is configured to detect the plurality of inner rings and generate a mask that approximates the inner rings.

20. The system of claim 19, wherein the first machine learning model is configured to detect the outer ring by segmenting the image into multiple patches and distinguishing the outer ring from background in each patch.
